SEQUENCE LISTING (14 sequences)

SEQ ID NO: 1 - protein, 5 aa, Artificial Sequence
  Cleavage site (EKsite) for enterokinase (rEK)
DDDDK

SEQ ID NO: 2 - DNA, 4106 bp, Artificial Sequence
  Plasmid pAMK024 used for integration, zeocin selection and recombinant expression of the chimeric iLOV-GSlinker-EKsite-NI01 protein in P. pastoris.
aacatccaaagacgaaaggttgaatgaaacctttttgccgacatccacaggtccattctcacacataagtgccaaacgcaacaggaggggatacactagcagcagaccgttgcaaacgcaggacctccactcctcttctcctcaacacccacttttgccatcgaaaaaccagcccagttattgggcttgattggagctcgctcattccaattccttctattaggctactaacaccatgactttattagcctgtctatcctggcccccctggcgaggttcatgtttgtttatttccgaatgcaacaagctccgcattacacccgaacatcactccagatgagggctttctgagtgtggggtcaaatagtttcatgttccccaaatggcccaaaactgacagtttaaacgctgtcttggaacctaatatgacaaaagcgtgatctcatccaagatgaactaagtttggttcgttgaaatgctaacggccagttggtcaaaaagaaacttccaaaagtcggcataccgtttgtcttgtttggtattgattgacgaatgctcaaaaataatctcattaatgcttagcgcagtctctctatcgcttctgaaccccggtgcacctgtgccgaaacgcaaatggggaaacacccgctttttggatgattatgcattgtctccacattgtatgcttccaagattctggtgggaatactgctgatagcctaacgttcatgatcaaaatttaactgttctaacccctacttgacagcaatatataaacagaaggaagctgccctgtcttaaacctttttttttatcatcattattagcttactttcataattgcgactggttccaattgacaagcttttgattttaacgacttttaacgacaacttgagaagatcaaaaaacaactaattattgaaagaattccgaaacgatgggccaccaccaccaccaccaccaccacatggcaactacacttgagagaattgagaaaaactttgttattactgaccctagacttccagacaacccaattatttttgcttctgatggtttcttggaattgactgagtactctagagaagagattttgggtagaaatgctagatttttgcaaggtcctgaaactgatcaagctactgttcaaaagattagagatgctatcagagatcaaagagagactactgttcaattgattaactacactaagtctggtaaaaagttctggaatttgttgcatttgcaaccagttagagatcaaaagggtgaattgcaatacttcatcggtgttcaattggatggtactgagcacgttggttctggttctggttctggagatgatgatgataagatggctgcttttatgaagttgatccaattcttggctactaagggtcaaaaatatgtttctttggcttggaagcataaaggtactattttgaaatggatcaacgcaggacagtcattcgagtggatctacaagcagattaagaaactttgggcataggcggccgctcaagaggatgtcagaatgccatttgcctgagagatgcaggcttcatttttgatacttttttatttgtaacctatatagtataggattttttttgtcattttgtttcttctcgtacgagcttgctcctgatcagcctatctcgcagcagatgaatatcttgtggtaggggtttgggaaaatcattcgagtttgatgtttttcttggtatttcccactcctcttcagagtacagaagattaagtgagaccttcgtttgtgcggatccttcagtaatgtcttgtttcttttgttgcagtggtgagccattttgacttcgtgaaagtttctttagaatagttgtttccagaggccaaacattccacccgtagtaaagtgcaagcgtaggaagaccaagactggcataaatcaggtataagtgtcgagcactggcaggtgatcttctgaaagtttctactagcagataagatccagtagtcatgcatatggcaacaatgtaccgtgtggatctaagaacgcgtcctactaaccttcgcattcgttggtccagtttgttgttatcgatcaacgtgacaaggttgtcgattccgcgtaagcatgcatacccaaggacgcctgttgcaattccaagtgagccagttccaacaatctttgtaatattagagcacttcattgtgttgcgcttgaaagtaaaatgcgaacaaattaagagataatctcgaaaccgcgacttcaaacgccaatatgatgtgcggcacacaataagcgttcatatccgctgggtgactttctcgctttaaaaaattatccgaaaaaattttctagagtgttgttactttatacttccggctcgtataatacgacaaggtgtaaggaggactaaaccatggctaaactcacctctgctgttccagtcctgactgctcgtgatgttgctggtgctgttgagttctggactgatagactcggtttctcccgtgacttcgtagaggacgactttgccggtgttgtactgacgacgttaccctgttcatctccgcagttcaggaccaggttgtgccagacaacactctggcatgggtatgggttcgtggtctggacgaactgtacgctgagtggtctgaggtcgtgtctaccaacttccgtgatgcatctggtccagctatgaccgagatcggtgaacagccctggggtcgtgagtttgcactgcgtgatccagctggtaactgcgtgcatttcgtcgcagaagagcaggactaacaattgacaccttacgattatttagagagtatttattagttttattgtatgtatacggatgttttattatctattagcccttatattctgtaactatccaaaagtcctatcttatcaagccagcaatctatgtccgcgaacgtcaactaaaaataagctttttatgctcttctctctttttttcccttcggtataattataccttgcatccacagattctcctgccaaattttgcataatcctttacaacatggctatatgggagcacttagcgccctccaaaacccatattgcctacgcatgtataggtgttttttccacaatattttctctgtgctctctttttattaaagagaagctctatatcggagaagcttctgtggccgttatattcggccttatcgtgggaccacattgcctgaattggtttgccccggaagattggggaaacttggatctgattaccttagctgcaggtaccactgagcgtcagaccccgtagaaaagatcaaaggatcttcttgagatcctttttttctgcgcgtaatctgctgcttgcaaacaaaaaaaccaccgctaccagcggtggtttgtttgccgatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcggtacccagatccaattcccgctttgactgcctgaaatctccatcgcctacaatgatgacatttggatttggttgactcatgttggtattgtgaaatagacgcagatcgggaacactgaaaaatacacagttattattcatttaaat

SEQ ID NO: 3 - protein, 190 aa, Artificial Sequence
  Chimeric iLOV, GS linker, EKsite and Staphylococcus capitis TE8 protein sequence
MGHHHHHHHHMATTLERIEKNFVITDPRLPDNPIIFASDGFLELTEYSREEILGRNARFLQGPETDQATVQKIRDAIRDQRETTVQLINYTKSGKKFWNLLHLQPVRDQKGELQYFIGVQLDGTEHVGSGSGSGDDDDKMAGFMKLIQFLATKGQKYVSLAWKHKGTILKWINAGQSFEWIYKQIKKLWS

SEQ ID NO: 4 - protein, 192 aa, Artificial Sequence
  Chimeric iLOV, GS linker, EKsite and Lactococcus lactis LacQ protein sequence.
MGHHHHHHHHMATTLERIEKNFVITDPRLPDNPIIFASDGFLELTEYSREEILGRNARFLQGPETDQATVQKIRDAIRDQRETTVQLINYTKSGKKFWNLLHLQPVRDQKGELQYFIGVQLDGTEHVGSGSGSGDDDDKMAGFLKVVQLLAKYGSKAVQWAWANKGKILDWLNAGQAIDWVVSKIKQILGIK

SEQ ID NO: 5 - protein, 190 aa, Artificial Sequence
  Chimeric iLOV, GS linker, EKsite and Staphylococcus aureus A53 protein sequence.
MGHHHHHHHHMATTLERIEKNFVITDPRLPDNPIIFASDGFLELTEYSREEILGRNARFLQGPETDQATVQKIRDAIRDQRETTVQLINYTKSGKKFWNLLHLQPVRDQKGELQYFIGVQLDGTEHVGSGSGSGDDDDKMSWLNFLKYIAKYGKKAVSAAWKYKGKVLEWLNVGPTLEWVWQKLKKIAGL

SEQ ID NO: 6 - protein, 176 aa, Artificial Sequence
  Chimeric iLOV, GS linker, EKsite and Homo sapiens LL-37 protein sequence.
MGHHHHHHHHMATTLERIEKNFVITDPRLPDNPIIFASDGFLELTEYSREEILGRNARFLQGPETDQATVQKIRDAIRDQRETTVQLINYTKSGKKFWNLLHLQPVRDQKGELQYFIGVQLDGTEHVGSGSGSGDDDDKLLGDFFRKSKEKIGKEFKRIVQRIKDFLRNLVPRTES

SEQ ID NO: 7 - protein, 163 aa, Artificial Sequence
  Chimeric iLOV, GS linker, EKsite and Homo sapiens His5 protein sequence.
MGHHHHHHHHMATTLERIEKNFVITDPRLPDNPIIFASDGFLELTEYSREEILGRNARFLQGPETDQATVQKIRDAIRDQRETTVQLINYTKSGKKFWNLLHLQPVRDQKGELQYFIGVQLDGTEHVGSGSGSGDDDDKDSHAKRHHGYKRKFHEKHHSHRGY

SEQ ID NO: 8 - protein, 186 aa, Artificial Sequence
  Chimeric iLOV, GS linker, EKsite and Homo sapiens DCD-1L protein sequence.
MGHHHHHHHHMATTLERIEKNFVITDPRLPDNPIIFASDGFLELTEYSREEILGRNARFLQGPETDQATVQKIRDAIRDQRETTVQLINYTKSGKKFWNLLHLQPVRDQKGELQYFIGVQLDGTEHVGSGSGSGDDDDKSSLLEKGLDGAKKAVGGLGKLGKDAVEDLESVGKGAVHDVKDLDSVL

SEQ ID NO: 9 - protein, 159 aa, Artificial Sequence
  Chimeric iLOV, GS linker, EK site and Podisus maculiventris Thn1 protein sequence.
MGHHHHHHHHMATTLERIEKNFVITDPRLPDNPIIFASDGFLELTEYSREEILGRNARFLQGPETDQATVQKIRDAIRDQRETTVQLINYTKSGKKFWNLLHLQPVRDQKGELQYFIGVQLDGTEHVGSGSGSDDDDKGSKKPVPIIYCNRRTGKCQRM

SEQ ID NO: 10 - DNA, 4879 bp, Artificial Sequence
  Plasmid pCVD002 used for integration, zeocin selection, recombinant expression and secretion of the chimeric iLOV-GSlinker-EKsite-SpyTag-SARS-CoV-2-RBD protein in P. pastoris.
cgaaacgatgagattcccatctattttcaccgctgtcttgttcgctgcctcctctgcattggctgcccctgttaacactaccactgaagacgagactgctcaaattccagctgaagcagttatcggttactctgaccttgagggtgatttcgacgtcgctgttttgcctttctctaactccactaacaacggtttgttgttcattaacaccactatcgcttccattgctgctaaggaagagggtgtctctctcgagaagagacaccatcatcatcatcatcatcatatggcaactacacttgagagaattgagaaaaactttgttattactgaccctagacttccagacaacccaattatttttgcttctgatggtttcttggaattgactgagtactctagagaagagattttgggtagaaatgctagatttttgcaaggtcctgaaactgatcaagctactgttcaaaagattagagatgctatcagagatcaaagagagactactgttcaattgattaactacactaagtctggtaaaaagttctggaatttgttgcatttgcaaccagttagagatcaaaagggtgaattgcaatacttcatcggtgttcaattggatggtactgagcacgttggttctggttctggttctggagatgatgatgataaggcccacattgtgatggttgatgcttataagccaacaaaaggttcaggaggaagcggtggttcaggtacaggcaacattaccaatttgtgcccatttggtgaagtgttcaatgctactagatttgcctctgtttacgcttggaatagaaagaggatctctaactgtgttgctgattattcggtcttgtacaattcagcaagtttcagtaccttcaagtgttacggagtttctcctacaaaactgaacgacttgtgctttacgaatgtttatgctgactcttttgtcattagaggagatgaagttagacagatagcaccaggtcaaacgggtaaaatagccgattacaactacaagttgcctgatgacttcactggatgtgtcattgcctggaatagcaacaatcttgattccaaagtaggtgggaactacaactatctatatcgtctgttccgtaaatccaacttaaagccctttgagagagacatcagtactgagatctaccaagctggatctaccccttgtaatggcgttgaaggtttcaactgctactttccgttacagtcgtatgggtttcaacccactaatggagttggctatcaaccatatcgagtcgtagtgttgtcctttgagctacttcatgcaccagctactgtatgtggtcctaagaaataagcggccgctcaagaggatgtcagaatgccatttgcctgagagatgcaggcttcatttttgatacttttttatttgtaacctatatagtataggattttttttgtcattttgtttcttctcgtacgagcttgctcctgatcagcctatctcgcagcagatgaatatcttgtggtaggggtttgggaaaatcattcgagtttgatgtttttcttggtatttcccactcctcttcagagtacagaagattaagtgagaccttcgtttgtgcggatccttcagtaatgtcttgtttcttttgttgcagtggtgagccattttgacttcgtgaaagtttctttagaatagttgtttccagaggccaaacattccacccgtagtaaagtgcaagcgtaggaagaccaagactggcataaatcaggtataagtgtcgagcactggcaggtgatcttctgaaagtttctactagcagataagatccagtagtcatgcatatggcaacaatgtaccgtgtggatctaagaacgcgtcctactaaccttcgcattcgttggtccagtttgttgttatcgatcaacgtgacaaggttgtcgattccgcgtaagcatgcatacccaaggacgcctgttgcaattccaagtgagccagttccaacaatctttgtaatattagagcacttcattgtgttgcgcttgaaagtaaaatgcgaacaaattaagagataatctcgaaaccgcgacttcaaacgccaatatgatgtgcggcacacaataagcgttcatatccgctgggtgactttctcgctttaaaaaattatccgaaaaaattttctagagtgttgttactttatacttccggctcgtataatacgacaaggtgtaaggaggactaaaccatggctaaactcacctctgctgttccagtcctgactgctcgtgatgttgctggtgctgttgagttctggactgatagactcggtttctcccgtgacttcgtagaggacgactttgccggtgttgtacgtgacgacgttaccctgttcatctccgcagttcaggaccaggttgtgccagacaacactctggcatgggtatgggttcgtggtctggacgaactgtacgctgagtggtctgaggtcgtgtctaccaacttccgtgatgcatctggtccagctatgaccgagatcggtgaacagccctggggtcgtgagtttgcactgcgtgatccagctggtaactgcgtgcatttcgtcgcagaagagcaggactaacaattgacaccttacgattatttagagagtatttattagttttattgtatgtatacggatgttttattatctatttatgcccttatattctgtaactatccaaaagtcctatcttatcaagccagcaatctatgtccgcgaacgtcaactaaaaataagctttttatgctcttctctctttttttcccttcggtataattataccttgcatccacagattctcctgccaaattttgcataatcctttacaacatggctatatgggagcacttagcgccctccaaaacccatattgcctacgcatgtataggtgttttttccacaatattttctctgtgctctctttttattaaagagaagctctatatcggagaagcttctgtggccgttatattcggccttatcgtgggaccacattgcctgaattggtttgccccggaagattggggaaacttggatctgattaccttagctgcaggtaccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcggtacccagatccaattcccgctttgactgcctgaaatctccatcgcctacaatgatgacatttggatttggttgactcatgttggtattgtgaaatagacgcagatcgggaacactgaaaaatacacagttattattcatttaaataacatccaaagacgaaaggttgaatgaaacctttttgccatccgacatccacaggtccattctcacacataagtgccaaacgcaacaggaggggatacactagcagcagaccgttgcaaacgcaggacctccactcctcttctcctcaacacccacttttgccatcgaaaaaccagcccagttattgggcttgattggagctcgctcattccaattccttctattaggctactaacaccatgactttattagcctgtctatcctggcccccctggcgaggttcatgtttgtttatttccgaatgcaacaagctccgcattacacccgaacatcactccagatgagggctttctgagtgtggggtcaaatagtttcatgttccccaaatggcccaaaactgacagtttaaacgctgtcttggaacctaatatgacaaaagcgtgatctcatccaagatgaactaagtttggttcgttgaaatgctaacggccagttggtcaaaaagaaacttccaaaagtcggcataccgtttgtcttgtttggtattgattgacgaatgctcaaaaataatctcattaatgcttagcgcagtctctctatcgcttctgaaccccggtgcacctgtgccgaaacgcaaatggggaaacacccgctttttggatgattatgcattgtctccacattgtatgcttccaagattctggtgggaatactgctgatagcctaacgttcatgatcaaaatttaactgttctaacccctacttgacagcaatatataaacagaaggaagctgccctgtcttaaacctttttttttatcatcattattagcttactttcataattgcgactggttccaattgacaagcttttgattttaacgacttttaacgacaacttgagaagatcaaaaaacaactaattattgaaagaattc

SEQ ID NO: 11 - DNA, 4867 bp, Artificial Sequence
  Plasmid pCVD005 used for integration, zeocin selection, recombinant expression and secretion of the chimeric iLOV-GSlinker-EKsite-SARS-CoV-2-RBD-SpyTag protein in P. pastoris.
cgaaacgatgagattcccatctattttcaccgctgtcttgttcgctgcctcctctgcattggctgcccctgttaacactaccactgaagacgagactgctcaaattccagctgaagcagttatcggttactctgaccttgagggtgatttcgacgtcgctgttttgcctttctctaactccactaacaacggtttgttgttcattaacaccactatcgcttccattgctgctaaggaagagggtgtctctctcgagaagagacaccatcatcatcatcatcatcatatggcaactacacttgagagaattgagaaaaactttgttattactgaccctagacttccagacaacccaattatttttgcttctgatggtttcttggaattgactgagtactctagagaagagattttgggtagaaatgctagatttttgcaaggtcctgaaactgatcaagctactgttcaaaagattagagatgctatcagagatcaaagagagactactgttcaattgattaactacactaagtctggtaaaaagttctggaatttgttgcatttgcaaccagttagagatcaaaagggtgaattgcaatacttcatcggtgttcaattggatggtactgagcacgttggttctggttctggttctggagatgatgatgataagaatatcaccaatttgtgtccctttggtgaggttttcaatgcaaccagatttgcctccgtttatgcatggaacagaaagcgtatttccaactgtgttgctgattactcagttttgtacaatagtgcctcattttccacgttcaaatgctacggtgtaagtccaactaagttgaacgacctatgctttaccaatgtctacgctgattctttcgtgattagaggagatgaagtccgacaaattgctcctggtcaaactggcaaaatcgctgattacaactacaagttaccagatgactttactggatgtgtgattgcctggaactctaataacctggactctaaagttggtggtaactacaattacctgtatcgtcttttcaggaaaagcaaccttaagccgtttgaaagagacataagcacagagatctatcaagctggatcaacaccttgtaatggagtggaagggttcaactgctatttcccattgcagtcttatggctttcaacctacgaatggtgtaggctatcagccttatagagttgtagtcttatcgtttgagttgctacatgctccagcaactgtttgtggaccaaagaaaacagggagtggaggttctggtgctcacatagtcatggttgatgcctataaacccactaagtaagcggccgctcaagaggatgtcagaatgccatttgcctgagagatgcaggcttcatttttgatacttttttatttgtaacctatatagtataggattttttttgtcattttgtttcttctcgtacgagcttgctcctgatcagcctatctcgcagcagatgaatatcttgtggtaggggtttgggaaaatcattcgagtttgatgtttttcttggtatttcccactcctcttcagagtacagaagattaagtgagaccttcgtttgtgcggatccttcagtaatgtcttgtttcttttgttgcagtggtgagccattttgacttcgtgaaagtttctttagaatagttgtttccagaggccaaacattccacccgtagtaaagtgcaagcgtaggaagaccaagactggcataaatcaggtataagtgtcgagcactggcaggtgatcttctgaaagtttctactagcagataagatccagtagtcatgcatatggcaacaatgtaccgtgtggatctaagaacgcgtcctactaaccttcgcattcgttggtccagtttgttgttatcgatcaacgtgacaaggttgtcgattccgcgtaagcatgcatacccaaggacgcctgttgcaattccaagtgagccagttccaacaatctttgtaatattagagcacttcattgtgttgcgcttgaaagtaaaatgcgaacaaattaagagataatctcgaaaccgcgacttcaaacgccaatatgatgtgcggcacacaataagcgttcatatccgctgggtgactttctcgctttaaaaaattatccgaaaaaattttctagagtgttgttactttatacttccggctcgtataatacgacaaggtgtaaggaggactaaaccatggctaaactcacctctgctgttccagtcctgactgctcgtgatgttgctggtgctgttgagttctggactgatagactcggtttctcccgtgacttcgtagaggacgactttgccggtgttgtacgtgacgacgttaccctgttcatctccgcagttcaggaccaggttgtgccagacaacactctggcatgggtatgggttcgtggtctggacgaactgtacgctgagtggtctgaggtcgtgtctaccaacttccgtgatgcatctggtccagctatgaccgagatcggtgaacagccctggggtcgtgagtttgcactgcgtgatccagctggtaactgcgtgcatttcgtcgcagaagagcaggactaacaattgacaccttacgattatttagagagtatttattagttttattgtatgtatacggatgttttattatctatttatgcccttatattctgtaactatccaaaagtcctatcttatcaagccagcaatctatgtccgcgaacgtcaactaaaaataagctttttatgctcttctctctttttttcccttcggtataattataccttgcatccacagattctcctgccaaattttgcataatcctttacaacatggctatatgggagcacttagcgccctccaaaacccatattgcctacgcatgtataggtgttttttccacaatattttctctgtgctctctttttattaaagagaagctctatatcggagaagcttctgtggccgttatattcggccttatcgtgggaccacattgcctgaattggtttgccccggaagattggggaaacttggatctgattaccttagctgcaggtaccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcggtacccagatccaattcccgctttgactgcctgaaatctccatcgcctacaatgatgacatttggatttggttgactcatgttggtattgtgaaatagacgcagatcgggaacactgaaaaatacacagttattattcatttaaataacatccaaagacgaaaggttgaatgaaacctttttgccatccgacatccacaggtccattctcacacataagtgccaaacgcaacaggaggggatacactagcagcagaccgttgcaaacgcaggacctccactcctcttctcctcaacacccacttttgccatcgaaaaaccagcccagttattgggcttgattggagctcgctcattccaattccttctattaggctactaacaccatgactttattagcctgtctatcctggcccccctggcgaggttcatgtttgtttatttccgaatgcaacaagctccgcattacacccgaacatcactccagatgagggctttctgagtgtggggtcaaatagtttcatgttccccaaatggcccaaaactgacagtttaaacgctgtcttggaacctaatatgacaaaagcgtgatctcatccaagatgaactaagtttggttcgttgaaatgctaacggccagttggtcaaaaagaaacttccaaaagtcggcataccgtttgtcttgtttggtattgattgacgaatgctcaaaaataatctcattaatgcttagcgcagtctctctatcgcttctgaaccccggtgcacctgtgccgaaacgcaaatggggaaacacccgctttttggatgattatgcattgtctccacattgtatgcttccaagattctggtgggaatactgctgatagcctaacgttcatgatcaaaatttaactgttctaacccctacttgacagcaatatataaacagaaggaagctgccctgtcttaaacctttttttttatcatcattattagcttactttcataattgcgactggttccaattgacaagcttttgattttaacgacttttaacgacaacttgagaagatcaaaaaacaactaattattgaaagaattc

SEQ ID NO: 12 - DNA, 2896 bp, Artificial Sequence
  Plasmid pAMK051 encoding iLOV under control of the DcuB promotor (pDcuB).
atgactgaaatgcctcaaaatgttctttacgatgccattgggatatatcaacggtggtatatccagtgatttttttctccattttagcttccttagctcctgaaaatctcgataactcaaaaaatacgcccggtagtgatcttatttcattatggtgaaagttggaacctcttacgtgccgatcaacgtctcattttcgccaaaagttggcccagggcttcccggtatcaacagggacaccaggatttatttattctgcgaagtgatcttccgtcacaggtatttattcggcgcaaagtgcgtcgggtgatgctgccaacttactgatttagtgtatgatggtgtttttgaggtgctccagtggcttctgtttctatcagctgtccctcctgttcagctactgacggggtggtgcgtaacggcaaaagcaccgccggacatcagcgctagcggagtgtatactggcttactatgttggcactgatgagggtgtcagtgaagtgcttcatgtggcaggagaaaaaaggctgcaccggtgcgtcagcagaatatgtgatacaggatatattccgcttcctcgctcactgactcgctacgctcggtcgttcgactgcggcgagcggaaatggcttacgaacggggcggagatttcctggaagatgccaggaagatacttaacagggaagtgagagggccgcggcaaagccgtttttccataggctccgcccccctgacaagcatcacgaaatctgacgctcaaatcagtggtggcgaaacccgacaggactataaagataccaggcgtttccccctggcggctccctcgtgcgctctcctgttcctgcctttcggtttaccggtgtcattccgctgttatggccgcgtttgtctcattccacgcctgacactcagttccgggtaggcagttcgctccaagctggactgtatgcacgaaccccccgttcagtccgaccgctgcgccttatccggtaactatcgtcttgagtccaacccggaaagacatgcaaaagcaccactggcagcagccactggtaattgatttagaggagttagtcttgaagtcatgcgccggttaaggctaaactgaaaggacaagttttggtgactgcgctcctccaagccagttacctcggttcaaagagttggtagctcagagaaccttcgaaaaaccgccctgcaaggcggttttttcgttttcagagcaagagattacgcgcagaccaaaacgatctcaagaagatcatcttattaatcagataaaatatttctagatttcagtgcaatttatctcttcaaatgtagcacctgaagtcagcctgtgctcggatcgctggttatctgtaagtaataattattaccgtaatgcaaaatacccctggcacaagttacatccttcatcagtaaaacttgtgccagatcaaatataattatccctccatcatcgctaaaaattaatatctcttcaggtgaacggtgtttttaatttcaaaacgctaacaaaagttaattaactattatgtcacccgcattatgtgtatttttacccacaaatgggtagatcagattaatctataaacctaatgacatctgccctgagaacaaaaaatagaccgataaatatcaataagataacagcaaacaaaacattaacatctgcgcagtacaaactataaacccatcgccagagagtctttctctctgaaaaagccgcttatcacagtgcataaatttgccgctgctttaatcagccaatattcactgtgaggtatttgctaaagccggtaacgaccaaacggatatttagtcaggctctgaaaacagttcatacaaaacagaacgtgactgtgatctattcagcaaaaatttaaataggattatcgcgagggttcacatatggcaaccacactggaacgtatcgaaaaaaactttgttattaccgatccgcgtctgccggataatccgatcatttttgcaagtgatggttttctggaactgaccgaatatagccgtgaagaaattctgggtcgtaatgcacgttttctgcagggtcctgaaaccgatcaggcaaccgttcagaaaattcgtgatgccattcgtgatcagcgtgaaaccaccgttcagctgattaactataccaaaagcggcaaaaaattctggaatctgctgcatctgcagccggtgcgtgatcagaaaggtgaactgcagtattttatcggtgttcagctggatggcaccgaacatgtgtagagcgttctggcgctgggcgtttaagggcaccaataactgccttaaaaaaattacgccccgccctgccactcatcgcagtactgttgtaattcattaagcattctgccgacatggaagccatcacagacggcatgatgaacctgaatcgccagcggcatcagcaccttgtcgccttgcgtataatatttgcccatggtgaaaacgggggcgaagaagttgtccatattggccacgtttaaatcaaaactggtgaaactcacccagggattggctgagacgaaaaacatattctcaataaaccctttagggaaataggccaggttttcaccgtaacacgccacatcttgcgaatatatgtgtagaaactgccggaaatcgtcgtggtattcactccagagcgatgaaaacgtttcagtttgctcatggaaaacggtgtaacaagggtgaacactatcccatatcaccagctcaccgtctttcattgccatacggaattccggatgagcattcatcaggcgggcaagaatgtgaataaaggccggataaaacttgtgcttatttttctttacggtctttaaaaaggccgtaatatccagctgaacggtctggttataggtacattgagcaa

SEQ ID NO: 13 - DNA, 5424 bp, Artificial Sequence
  Plasmid pAMK052 encoding iLOV under the control of the DcuB promoter (pDcuB) and DcuS/DcuR under the control of the DcuS promoter (pDcuS).
ctgactgaaatgcctcaaaatgttctttacgatgccattgggatatatcaacggtggtatatccagtgatttttttctccattttagcttccttagctcctgaaaatctcgataactcaaaaaatacgcccggtagtgatcttatttcattatggtgaaagttggaacctcttacgtgccgatcaacgtctcattttcgccaaaagttggcccagggcttcccggtatcaacagggacaccaggatttatttattctgcgaagtgatcttccgtcacaggtatttattcggcgcaaagtgcgtcgggtgatgctgccaacttactgatttagtgtatgatggtgtttttgaggtgctccagtggcttctgtttctatcagctgtccctcctgttcagctactgacggggtggtgcgtaacggcaaaagcaccgccggacatcagcgctagcggagtgtatactggcttactatgttggcactgatgagggtgtcagtgaagtgcttcatgtggcaggagaaaaaaggctgcaccggtgcgtcagcagaatatgtgatacaggatatattccgcttcctcgctcactgactcgctacgctcggtcgttcgactgcggcgagcggaaatggcttacgaacggggcggagatttcctggaagatgccaggaagatacttaacagggaagtgagagggccgcggcaaagccgtttttccataggctccgcccccctgacaagcatcacgaaatctgacgctcaaatcagtggtggcgaaacccgacaggactataaagataccaggcgtttccccctggcggctccctcgtgcgctctcctgttcctgcctttcggtttaccggtgtcattccgctgttatggccgcgtttgtctcattccacgcctgacactcagttccgggtaggcagttcgctccaagctggactgtatgcacgaaccccccgttcagtccgaccgctgcgccttatccggtaactatcgtcttgagtccaacccggaaagacatgcaaaagcaccactggcagcagccactggtaattgatttagaggagttagtcttgaagtcatgcgccggttaaggctaaactgaaaggacaagttttggtgactgcgctcctccaagccagttacctcggttcaaagagttggtagctcagagaaccttcgaaaaaccgccctgcaaggcggttttttcgttttcagagcaagagattacgcgcagaccaaaacgatctcaagaagatcatcttattaatcagataaaatatttctagatttcagtgcaatttatctcttcaaatgtagcacctgaagtcagcctgtgctcttattggcaatattgtttcagtagtgagtagtgttctgcctgaatacggtaacggtaaactggacgccccgtgacgccataatggatactggtgaacaagatgtggcagttgaccagccagatgaggtatttacggcaggaaacacgcgaaatgttaacctcgttggctagctcgtcggttgaaaattcatagtcctgatgcgcgtcaatccactggcacagtgtgcgtaacgtctgcggcgttaagccttttggcaagcgacgaggatcctgttcgttggagctgctgccgtggattagctgatcaagctcggcctggtcataatactgatgtttttccagcgccattttcttttgccgccagccggtgagcgcctcttcaaagcgggaagcctggaagggtttgatcaggtaatccacgacaccgtaatgcagcgaatctttaatggttgccgcatcggctgcggaggagatgacaatcacatcacttttgcaacgcgcgttatgcaggacaggcagtaaatcgagcccgttctctttttgcatatagatatcgagcaatatcaggtcgataggcgtatcgctattgaagataatctctttggctttctccagcgtcgaggctgttccacagcattgaaagcctgggatttgtgctacgtatcggcgattcagctccgcgaccattgcgtcgtcatcgataattaatacattgatcatctgttcgacctctccccgtcccagggtatctggacaaaaaattgtgtgaaaatcccgggttccgattccacggcgatgctgccgccgagattttctacctgttgtttgacaagtgctaaaccgacgcctcgctcgcttccttttgtcgagacacctttgtcaaaaatgtgatcgattttatcgggtgcgatccccggtccatcatcattaacttcacagtgcagccagccgtgacggtagtgcaatgttacgctaatttcgcctccgggttccggccctaatgcctccagcgcgttttctatcagatttcccaacgtggtaatcagcgtcgcgacctggtcctcactgccgctgtctggcagctggctttcactgtttaaaatcagcgtatggcctaaatcggtcgcgcggttaatcttgctgattaaaaaaccagcgataaccggagatttgatcttacccagcagagagccaatctcttcctgatagttattggctgttttgagaatgtaatcttccaactgcttataactcttcagatgcaataatccgagaatcacatgcaatttattcataaattcgtgggatcgttcacgaagtgcgtcagcatagttgaccagaccgtcgagtcgctgcatcagtttacgtacttcagttttgtccctgaaggttgaaatggcaccgatgataacgccattactgcgcaccggaacggtgttgatcagtaatagccggtctttaatcgtaatctcttcgtcgcggcgcggggtaccgtcgcgtaacacttccgagacatctaccacctgtgaccatgagtggcttagcgtcgacagtttctcatcgtcctgcgacttacggtaattcagcaattcttgtgcggcatcgttgatcagcgtgacctcgccgcgatcgtccacggcaacgacgccttctttgatagactgcaacatggcctggcgttgctcaaacagcgtggagatttcgtagggttccaggccgaaaaggatttttttcagtaccttaaccagaatgcaggtgccaatcagtccgaccagcatgccaaataataccgaccagataatgctccagcgactgtcattgatctgttgggtcacacggcttaactcaaggccgatcgccaccacgccaatttgtttatgattttcatcgtagatgggggtaaatacgcgtaaagcctgcgccagaaaaccgcgattgatagcgacattttcttcgccattcagcgctttaaggatgtcatcacctttaaatggctgaccaatacgctgggcttcaggatgcgagtagcgaagactttgcatatcggtaacgacaataaacagcagatcgttgcgtttgcgtacggcttccgcgatggcctggatgccactctcctgcggttttttctgcaagccctgacggatttccggcgagtcggcgagggtacgcgccactgccagtgccttgttggctagcccatctcgcgtcatatcactgatttgcgagaagtaaatcagatgcaccaccaatagcaccgagaacagtaccgcactgaccattaagatcactgtggtactcaatttcatcggacgtttgcgtaacatgcggtagggcaatgaatgtctcatcagcttccttgtgtgacaaatttcttaagcattatctctgatgaggcgggtaattcaaagggagtaagaatgattggctatataggggaagagactctggcaacggaaactgccagtgctgtatgaagattccggggctatgcttatagcgataatcatactgatgagagagggaaggtcggatcgctggttatctgtaagtaataattattaccgtaatgcaaaatacccctggcacaagttacatccttcatcagtaaaacttgtgccagatcaaatataattatccctccatcatcgctaaaaattaatatctcttcaggtgaacggtgtttttaatttcaaaacgctaacaaaagttaattaactattatgtcacccgcattatgtgtatttttacccacaaatgggtagatcagattaatctataaacctaatgacatctgccctgagaacaaaaaatagaccgataaatatcaataagataacagcaaacaaaacattaacatctgcgcagtacaaactataaacccatcgccagagagtctttctctctgaaaaagccgcttatcacagtgcataaatttgccgctgctttaatcagccaatattcactgtgaggtatttgctaaagccggtaacgaccaaacggatatttagtcaggctctgaaaacagttcatacaaaacagaacgtgactgtgatctattcagcaaaaatttaaataggattatcgcgagggttcacatatggcaaccacactggaacgtatcgaaaaaaactttgttattaccgatccgcgtctgccggataatccgatcatttttgcaagtgatggttttctggaactgaccgaatatagccgtgaagaaattctgggtcgtaatgcacgttttctgcagggtcctgaaaccgatcaggcaaccgttcagaaaattcgtgatgccattcgtgatcagcgtgaaaccaccgttcagctgattaactataccaaaagcggcaaaaaattctggaatctgctgcatctgcagccggtgcgtgatcagaaaggtgaactgcagtattttatcggtgttcagctggatggcaccgaacatgtgtagagcgttctggcgctgggcgtttaagggcaccaataactgccttaaaaaaattacgccccgccctgccactcatcgcagtactgttgtaattcattaagcattctgccgacatggaagccatcacagacggcatgatgaacctgaatcgccagcggcatcagcaccttgtcgccttgcgtataatatttgcccatggtgaaaacgggggcgaagaagttgtccatattggccacgtttaaatcaaaactggtgaaactcacccagggattggctgagacgaaaaacatattctcaataaaccctttagggaaataggccaggttttcaccgtaacacgccacatcttgcgaatatatgtgtagaaactgccggaaatcgtcgtggtattcactccagagcgatgaaaacgtttcagtttgctcatggaaaacggtgtaacaagggtgaacactatcccatatcaccagctcaccgtctttcattgccatacggaattccggatgagcattcatcaggcgggcaagaatgtgaataaaggccggataaaacttgtgcttatttttctttacggtctttaaaaaggccgtaatatccagctgaacggtctggttataggtacattgagcaa

SEQ ID NO: 14 - DNA, 5264 bp, Artificial Sequence
  Plasmid pAMK053 encoding the synthetic iLOV-DcuS-DcuR operon under the control of the DcuB promoter (pDcuB).
ctgactgaaatgcctcaaaatgttctttacgatgccattgggatatatcaacggtggtatatccagtgatttttttctccattttagcttccttagctcctgaaaatctcgataactcaaaaaatacgcccggtagtgatcttatttcattatggtgaaagttggaacctcttacgtgccgatcaacgtctcattttcgccaaaagttggcccagggcttcccggtatcaacagggacaccaggatttatttattctgcgaagtgatcttccgtcacaggtatttattcggcgcaaagtgcgtcgggtgatgctgccaacttactgatttagtgtatgatggtgtttttgaggtgctccagtggcttctgtttctatcagctgtccctcctgttcagctactgacggggtggtgcgtaacggcaaaagcaccgccggacatcagcgctagcggagtgtatactggcttactatgttggcactgatgagggtgtcagtgaagtgcttcatgtggcaggagaaaaaaggctgcaccggtgcgtcagcagaatatgtgatacaggatatattccgcttcctcgctcactgactcgctacgctcggtcgttcgactgcggcgagcggaaatggcttacgaacggggcggagatttcctggaagatgccaggaagatacttaacagggaagtgagagggccgcggcaaagccgtttttccataggctccgcccccctgacaagcatcacgaaatctgacgctcaaatcagtggtggcgaaacccgacaggactataaagataccaggcgtttccccctggcggctccctcgtgcgctctcctgttcctgcctttcggtttaccggtgtcattccgctgttatggccgcgtttgtctcattccacgcctgacactcagttccgggtaggcagttcgctccaagctggactgtatgcacgaaccccccgttcagtccgaccgctgcgccttatccggtaactatcgtcttgagtccaacccggaaagacatgcaaaagcaccactggcagcagccactggtaattgatttagaggagttagtcttgaagtcatgcgccggttaaggctaaactgaaaggacaagttttggtgactgcgctcctccaagccagttacctcggttcaaagagttggtagctcagagaaccttcgaaaaaccgccctgcaaggcggttttttcgttttcagagcaagagattacgcgcagaccaaaacgatctcaagaagatcatcttattaatcagataaaatatttctagatttcagtgcaatttatctcttcaaatgtagcacctgaagtcagcctgtgctcggatcgctggttatctgtaagtaataattattaccgtaatgcaaaatacccctggcacaagttacatccttcatcagtaaaacttgtgccagatcaaatataattatccctccatcatcgctaaaaattaatatctcttcaggtgaacggtgtttttaatttcaaaacgctaacaaaagttaattaactattatgtcacccgcattatgtgtatttttacccacaaatgggtagatcagattaatctataaacctaatgacatctgccctgagaacaaaaaatagaccgataaatatcaataagataacagcaaacaaaacattaacatctgcgcagtacaaactataaacccatcgccagagagtctttctctctgaaaaagccgcttatcacagtgcataaatttgccgctgctttaatcagccaatattcactgtgaggtatttgctaaagccggtaacgaccaaacggatatttagtcaggctctgaaaacagttcatacaaaacagaacgtgactgtgatctattcagcaaaaatttaaataggattatcgcgagggttcacatatggcaaccacactggaacgtatcgaaaaaaactttgttattaccgatccgcgtctgccggataatccgatcatttttgcaagtgatggttttctggaactgaccgaatatagccgtgaagaaattctgggtcgtaatgcacgttttctgcagggtcctgaaaccgatcaggcaaccgttcagaaaattcgtgatgccattcgtgatcagcgtgaaaccaccgttcagctgattaactataccaaaagcggcaaaaaattctggaatctgctgcatctgcagccggtgcgtgatcagaaaggtgaactgcagtattttatcggtgttcagctggatggcaccgaacatgtgtagtttgtcacacaaggaagctgatgagacattcattgccctaccgcatgttacgcaaacgtccgatgaaattgagtaccacagtgatcttaatggtcagtgcggtactgttctcggtgctattggtggtgcatctgatttacttctcgcaaatcagtgatatgacgcgagatgggctagccaacaaggcactggcagtggcgcgtaccctcgccgactcgccggaaatccgtcagggcttgcagaaaaaaccgcaggagagtggcatccaggccatcgcggaagccgtacgcaaacgcaacgatctgctgtttattgtcgttaccgatatgcaaagtcttcgctactcgcatcctgaagcccagcgtattggtcagccatttaaaggtgatgacatccttaaagcgctgaatggcgaagaaaatgtcgctatcaatcgcggttttctggcgcaggctttacgcgtatttacccccatctacgatgaaaatcataaacaaattggcgtggtggcgatcggccttgagttaagccgtgtgacccaacagatcaatgacagtcgctggagcattatctggtcggtattatttggcatgctggtcggactgattggcacctgcattctggttaaggtactgaaaaaaatccttttcggcctggaaccctacgaaatctccacgctgtttgagcaacgccaggccatgttgcagtctatcaaagaaggcgtcgttgccgtggacgatcgcggcgaggtcacgctgatcaacgatgccgcacaagaattgctgaattaccgtaagtcgcaggacgatgagaaactgtcgacgctaagccactcatggtcacaggtggtagatgtctcggaagtgttacgcgacggtaccccgcgccgcgacgaagagattacgattaaagaccggctattactgatcaacaccgttccggtgcgcagtaatggcgttatcatcggtgccatttcaaccttcagggacaaaactgaagtacgtaaactgatgcagcgactcgacggtctggtcaactatgctgacgcacttcgtgaacgatcccacgaatttatgaataaattgcatgtgattctcggattattgcatctgaagagttataagcagttggaagattacattctcaaaacagccaataactatcaggaagagattggctctctgctgggtaagatcaaatctccggttatcgctggttttttaatcagcaagattaaccgcgcgaccgatttaggccatacgctgattttaaacagtgaaagccagctgccagacagcggcagtgaggaccaggtcgcgacgctgattaccacgttgggaaatctgatagaaaacgcgctggaggcattagggccggaacccggaggcgaaattagcgtaacattgcactaccgtcacggctggctgcactgtgaagttaatgatgatggaccggggatcgcacccgataaaatcgatcacatttttgacaaaggtgtctcgacaaaaggaagcgagcgaggcgtcggtttagcacttgtcaaacaacaggtagaaaatctcggcggcagcatcgccgtggaatcggaacccgggattttcacacaattttttgtccagataccctgggacggggagaggtcgaacagatgatcaatgtattaattatcgatgacgacgcaatggtcgcggagctgaatcgccgatacgtagcacaaatcccaggctttcaatgctgtggaacagcctcgacgctggagaaagccaaagagattatcttcaatagcgatacgcctatcgacctgatattgctcgatatctatatgcaaaaagagaacgggctcgatttactgcctgtcctgcataacgcgcgttgcaaaagtgatgtgattgtcatctcctccgcagccgatgcggcaaccattaaagattcgctgcattacggtgtcgtggattacctgatcaaacccttccaggcttcccgctttgaagaggcgctcaccggctggcggcaaaagaaaatggcgctggaaaaacatcagtattatgaccaggccgagcttgatcagctaatccacggcagcagctccaacgaacaggatcctcgtcgcttgccaaaaggcttaacgccgcagacgttacgcacactgtgccagtggattgacgcgcatcaggactatgaattttcaaccgacgagctagccaacgaggttaacatttcgcgtgtttcctgccgtaaatacctcatctggctggtcaactgccacatcttgttcaccagtatccattatggcgtcacggggcgtccagtttaccgttaccgtattcaggcagaacactactcactactgaaacaatattgccaataaagcgttctggcgctgggcgtttaagggcaccaataactgccttaaaaaaattacgccccgccctgccactcatcgcagtactgttgtaattcattaagcattctgccgacatggaagccatcacagacggcatgatgaacctgaatcgccagcggcatcagcaccttgtcgccttgcgtataatatttgcccatggtgaaaacgggggcgaagaagttgtccatattggccacgtttaaatcaaaactggtgaaactcacccagggattggctgagacgaaaaacatattctcaataaaccctttagggaaataggccaggttttcaccgtaacacgccacatcttgcgaatatatgtgtagaaactgccggaaatcgtcgtggtattcactccagagcgatgaaaacgtttcagtttgctcatggaaaacggtgtaacaagggtgaacactatcccatatcaccagctcaccgtctttcattgccatacggaattccggatgagcattcatcaggcgggcaagaatgtgaataaaggccggataaaacttgtgcttatttttctttacggtctttaaaaaggccgtaatatccagctgaacggtctggttataggtacattgagcaa

(19) United States
(12) Patent Application Publication
(10) Pub. No.: US 2021/0206810 A1
(43) Pub. Date: Jul. 8, 2021

(54) Detection of Optimal Recombinants Using Fluorescent Protein Fusions

(71) Applicant: Ingenza Ltd., Roslin (GB)

(72) Inventors: Ian Fotheringham, Edinburgh (GB); Annemette Kjeldsen, Rosewell (GB); Leonardo Magneschi, Edinburgh (GB); Harveen Erskine, Penicuik (GB); Scott Baxter, Edinburgh (GB); Stephen McColm, Edinburgh (GB); Cristina Serrano-Amatriain, Edinburgh (GB); David McElroy, Edinburgh (GB); Jack Eric Kay, Musselburgh (GB)

(21) Appl. No.: 16/952,983

(22) Filed: Nov. 19, 2020

Related U.S. Application Data
(60) Provisional application No. 62/938,073, filed on Nov. 20, 2019.
(60) Provisional application No. 63/068,002, filed on Aug. 20, 2020.

(51) Int. Cl. C07K 14/005; C12N 15/62; C07K 14/47; C12N 7/00; C12P 21/00

(52) U.S. Cl. CPC C07K 14/005; C12N 15/62; C07K 14/47; C07K 14/4723; C12N 2770/18022; C12P 21/00; C07K 2319/50; C12N 2770/18052; C12N 2770/18023; C12N 7/00

(57) ABSTRACT

A detection of optimal genetic recombinants used to prepare target proteins, with assessment of their target-specific “upstream” productivity, genetic stability and means to optimize target protein “downstream” purification using customizable fluorescent tags. A scarless removable protein fusion makes it possible to identify recombinants of Pichia pastoris with optimal performance in heterologous protein production.

### RELATED APPLICATIONS

The present disclosure claims the filing priority of U.S. Provisional Application No. 62/938,073, titled “Detection of Optimal Recombinants Using Fluorescent Protein Fusions,” filed on Nov. 20, 2019, and U.S. Provisional Application No. 63/068,002, titled “Production of COVID-19 (SARS-CoV-2) Vaccine Component,” filed on Aug. 20, 2020. The '073 and '002 Provisional applications are hereby incorporated by reference.

### TECHNICAL FIELD OF THE INVENTION

The present invention relates to detection of optimal genetic recombinants used to prepare target proteins, with assessment of their target-specific “upstream” productivity, genetic stability and means to optimise target protein “downstream” purification using customisable fluorescent tags. More specifically, the invention relates to a scarless removable protein fusion to identify recombinants of a host with optimal performance in heterologous protein production.

### BACKGROUND OF THE INVENTION

Pichia pastoris is an industrially valuable yeast, used to produce heterologous proteins as protein therapeutics (biologics), recombinant subunit vaccine components, industrial biocatalysts or other applications. Unlike most other industrial hosts (e.g. E. coli, Bacillus, Saccharomyces) there are no plasmid vectors available for P. pastoris from which to express heterologous DNA introduced to the cell. Therefore, foreign DNA must integrate with the host genome to be stably maintained and expressed. While such genomic integration generally occurs, albeit with relatively low efficiency, this limitation means that, following transformation of P. pastoris with foreign DNA, there is genetic variability in the resulting transformants. Even if genomic integration of foe heterologous DNA is targeted to a specific genomic locus, it is common for multiple contiguous copies of the heterologous DNA to integrate either at the targeted locus or in single or multiple contiguous copies at random locations elsewhere in the host genome. Aberrant genomic recombination events also frequently occur, further increasing the heterogeneity of transformants. The high genetic diversity of transformants results in a corresponding high variability in foe expression level of foe integrated target gene between transformants. This necessitates extensive screening of transformants, by assay of the expressed target protein, to identify the most productive transformants. However, even when the most productive transformant is identified there is a major inherent problem. The more genomic copies of an integrated gene, generally the higher initial level of protein expression obtained. Vet, there is an inverse correlation between foe number of contiguous copies of an integrated target gene and the genetic stability of the recombinant strain, since intergenic recombination between identical contiguous heterologous DNA sequences can be highly favoured in P. pastoris. Therefore, recombinants isolated based on the highest initial productivity can also be those most likely to show genetic instability, whereby recombination events between contiguous identical copies of foe integrated target gene leads to removal (deletion) of gene copies with corresponding losses in productivity of the transformant to produce the target protein. Genetic instability is highly undesirable to the establishment of stable recombinant strains, particularly for pharmaceutical production to cGMP quality standards where strain stability is a priority. It is uncommon for a heterologous protein target of industrial or biomedical utility to also have a property that can be easily monitored in a recombinant yeast to readily assess the productivity and stability of the transformant. Generally, this would instead require complex and time consuming offline biochemical assays following culturing of the strain.

Isolation of the most productive yet stable transformants is very laborious, lengthy and highly unpredictable. Fusing a “reporter” protein to the target protein that can be detected colorimetrically, fluorescently or through other means drat report the level of production of the fusion is therefore attractive to monitor productivity and stability throughout bioprocess development and manufacturing of the target protein. However, rarely will a fused reporter protein attached to the target protein be acceptable to the end-user, particularly for pharmaceutical use. Therefore, any fusion protein must be separable without trace from the target protein following recovery and purification of the fusion from the culture of the recombinant production strain. This must also be cost-competitive, consistent and reproducible to be acceptable to the commercial end user, particularly for biomedical use. Chemical removal of the reporter and any linker amino acids is highly unattractive as it is sufficiently non-specific and harsh to likely damage the target protein. Enzymatic removal is limited as most proteases leave some residual amino acids from the linker on the target protein. Enterokinase is one enzyme that can remove ail protein sequence preceding or “upstream of” the target protein by locating its proteolytic cleavage site immediately adjacent to the N-terminus of the target protein. However, it is extremely expensive and not commercially available at scale. Therefore, for practical utility, the rapid identification of the most productive and stable P. pastoris transformants to express target proteins requires a readily detectable, low-burden, consistent and broadly useful reporter and a means, such as low cost enterokinase, to efficiently remove the reporter following recovery of the fusion protein from the culture. Until the invention of the present application, these and other problems in the prior art went cither unnoticed or unsolved by those skilled in the art.

Recombinant expression of biologically-active proteins can be challenging and highly influenced by both the physiology of the expression host and properties such as enzymatic activities or cytotoxic properties of the protein of interest, adding unpredictability to the success of bio-manufacturing processes and identification of the optimal recombinant strain. Fusing a “reporter” protein that is able to alter, diminish or prevent biological activity of the target protein is therefore attractive as a means to increase achievable product yield.

### SUMMARY OF THE INVENTION

The present application describes a novel means to isolate recombinants of the yeast Pichia pastoris with the highest and most genetically stable production of heterologous proteins. This is achieved by fusing a DNA sequence encoding the iLOV (Light-Oxygen-Voltage sensing) protein to a DNA sequence encoding the protein of interest to form a protein fusion. Fluorescence of the iLOV protein fusion in response to irradiation with blue light is directly proportionate to the level of expression of the protein fusion in each recombinant host cell, providing a visually or instrumentality detectable measurement of productivity between different recombinant hosts that can be quantified and tracked over a period. The fusion protein also includes a short protein (peptide) linker sequence between the iLOV and target proteins that can be specifically cleaved by the enzyme enterokinase (EK) to remove the entire iLOV and linker sequences, thereby leaving the target protein in its original unmodified form or “scarless”. The utility of this scarlessly removable fusion protein extends to “masking” undesired effects of the target protein such as toxicity to the host (or researchers) or the formation of unwanted aggregates that may reduce productivity of the host cell or activity of the heterologous protein.

These and other aspects of the invention may be understood more readily from the following description and the appended drawings.

### BRIEF DESCRIPTION OF THE DRAWINGS

For the purpose of facilitating an understanding of the subject matter sought to be protected, there are illustrated in the accompanying figures, embodiments thereof, from an inspection of which, when considered in connection with the following description, the subject matter sought to be protected, its construction and operation, and many of its advantages should be readily understood and appreciated.

FIG. 1 is a schematic of the iLOV-EK-N101 expression cassette used for recombinant intracellular expression in P. pastoris. This exemplary fusion consists of a polyhistidine-tagged iLOV with a C-terminal (GS)3-linker to the recognition sequence for enterokinase (rEK) (Asp-Asp-Asp-Asp-Lys)<SEQ. ID NO. 1>, followed by the 51 amino acid long N101 peptide.

FIG. 2 is an exemplary map and sequence of Plasmid pAMK24<SEQ. ID NO. 2>.

FIG. 3 is recombinant P. pastoris integrants expressing iLOV-EK-N101 visualised under daylight (left) and blue light (right; 302 nm) upon induction of the expression cassette on plates containing methanol.

FIG. 4a is a graph of normalised fluorescence values obtained from randomly selected integrants generated by transformation with iLOV-EK-N101 cassettes (either codon optimisation Opt1 or Opt2), grown in 24-well plates and induced with methanol for 48 hours.

FIG. 4b shows recombinant iLOV-EK-N101 protein expression in cell lysates predicted by fluorescence values (Ex450/Em500) as illustrated by Coomassie staining (top) and Western blotting (bottom; anti-iLOV antibody) of cell extracts from integrants highlighted in FIG. 4a. The expected iLOV-EK-N101 fusion MW, indicated by black arrow's, is 22 KDa; both protein loading and RFU are normalised by OD600).

FIG. 5 is a graph showing a fluorescence (530 nm emission) distribution of wildtype P. pastoris and positive control strain (+vc Cont) expressing iLOV-EK-N101. A clear difference between the two populations is observed.

FIG. 6 shows a fluorescence distribution of the ‘+ve Cont’ population and window P3 that was calibrated for sorting of the highest-expressing clones within a population of iLOV-EK-N101 integrants (e.g. ABP282 population, in the example).

FIG. 7 shows a validation of performances for P. pastoris integrants expressing iLOV-EK-N101 isolated by FACS. Clones were grown in liquid media and induced with methanol for 24 hours at small scale (24-well plates). Fluorescence is expressed as RFU/OD600 and values are reported relative to the ‘+ve Cont’ strain ABP269 (equal to 1).

FIG. 8 is a table showing yield improvement quantified through iLOV-EK-N101 fluorescence matching the percentage increase in purified N101 peptide. Clone ABP282-PL3-E12, showing highest fluorescence per OD600 at small scale, was assessed for performances at 100 mL scale relative to strain ABP269.

FIG. 9 shows expression of iLOV-EK-N101 and the reproducibility of two independent PreGMP fermentation runs as monitored following fluorescence.

FIG. 10 illustrates iLOV-EK-N101 fluorescence used for prompt detection of strain genetic instability and loss of integrated cassettes copies. Histograms indicate integrated copy numbers quantified via qPCR for iLOV sequence relative to two reference controls (HIS4 and ARG4, present in a single copy in the P. pastoris genome). Fluorescence values normalised by OD600 closely mirror integrated cassette copy numbers for pre-master cell bank #3 (pre-MCB3), research master cell bank #3 (rMCB3) and two different research working cell banks (rWCB3 and rWCB4).

FIG. 11 is a table showing fluorescence per OD600 at the end of fermentation (EoF) used to estimate productivity and subsequent yield of the iLOV-EK-N101 fusion and purified N101 peptide.

FIG. 12 is a Western blot analysis using anti-N101 antibodies to characterise the material obtained throughout the purification process. iLOV-EK-N101 fusion was first obtained via Immobilized Metal Ion Affinity Chromatography (IMAC). Cleavage with a low-cost recombinant enterokinase allows release of the native N101 sequence that can be subsequently separated from iLOV-EK and eluted at high purity. Black arrows indicate the expected molecular weights for each protein configuration.

FIG. 13 is a table showing minimum inhibitory concentration determined for iLOV-EK-N101 fusions and native N101, Growth of the susceptible organism Micrococcus luteus is inhibited (−) in presence of as low as 2 μg/mL N101, whereas iLOV-EK-N101 allows growth (+) even when it is added at 32 μg/mL, showing that iLOV-EK masks toxicity of the native peptide.

FIG. 14 is a table showing uncleaved iLOV-EK-N101 and iLOV-EK contaminants in purified N101 quantified through residual fluorescence. FMN is quantified by absorbance at 450 nm (FMN is the cofactor of iLOV, ratio 1:1) using extinction coefficient at 450 nm=12500 M⁻¹ cm⁻¹. Molar concentration calculated by the Beer-Lambert law: A=1cε. Protein concentration (mg/mL) is calculated from molar concentration using the formula mg/mL=M×MW (in Da). iLOV-EK-N101 and iLOV-EK impurities shown as off-target MW bands on SDS-PAGE gel stained with Coomassie and quantified using an imaging software closely match values obtained through absorbance.

FIG. 15 is a schematic of the iLOV-EK-AMP expression cassette used for recombinant expression of antimicrobial peptides (AMP) homologous to N101 in P. pastoris.

FIG. 16 shows iLOV-EK-AMP protein sequences for each of the fusions <SEQ. ID NOs. 3-9>; the AMP-specific aminoacidic sequence highlighted in grey was used to assess percent identity relative to NIDI.

FIG. 17 is a table showing the percent identity and predicted molecular weights (MW) for each of the iLOV-EK-AMP fusions of FIG. 16.

FIG. 18 shows clones isolated using the FACS approach always yield higher levels of fluorescence normalised by OD600 compared to randomly picked clones. Average RFU/OD600 values obtained after screening in liquid culture 10 randomly picked (white bars) versus 10 high-expressing clones selected by FACS (black bars) for each iLOV-EK-AMP-expressing P. pastoris strain are reported. The wildtype (-ve Ctrl) and a strain expressing iLOV-EK-N101 (N101) were included as controls (grey bars).

FIG. 19 shows protein expression levels for each of the iLOV-EK-AMP fusion strains correlate with their in vivo fluorescence values. Whole cell lysates of the highest (▴) and lowest (▾) fluorescence clones out of the top 10 identified by FACS were separated on SDS-PAGE and analysed by western blot using anti-iLOV antibodies.

FIG. 20 is a schematic of the iLOV-EK-SpyTag-SARS-CoV-2-RBD and iLOV-EK-SARS-CoV-2-RBD-SpyTag expression cassettes used for recombinant expression in P. pastoris. The pre-pro-α-factor secretion signal from S. cerevisiae was used for secretion of the protein fusions.

FIG. 21 is a maps and sequences of plasmids pCVD002<SEQ. ID NO. 10< and pCVD005<SEQ. ID NO. 11> as an example. N-terminal and C-terminal SpyTag fusions were generated.

FIG. 22 shows iLOV-EK-SpyTag-SARS-CoV-2-RBD fluorescence used to rank recombinant P. pastoris transformants for secretion at small scale (96-well plates). Culture fluorescence values were recorded after 48 h induction with methanol. Clones highlighted with black bars showed highest fluorescence levels and therefore secretion of the iLOV-EK-SpyTag-SARS-CoV-2-RBD fusion.

FIG. 23 shows the presence of a small fluorescent marker iLOV and the Enterokinase (EK) protease cleavage site at the N-terminus of SpyTag-SARS-CoV-2-RBD facilitates selection of high-yielding strains and release of the native SpyTag-SARS-CoV-2-RBD after cleavage with in-house rccombinantly produced Eft (see decrease in MW highlighted by black arrows). Western blot analysis using anti-SARS-CoV-2-RBD antibodies was used to characterise the material obtained throughout the purification process. The two N-linked glycosylation sites on the RBD displayed high mannose sugars as expected of protein secreted from Pichia. De-glycosylation of Expi293 (+vc CTRL) and Pichia-derived RBD with PNGase F generated a band recognised by anti-RBD antibodies at the expected molecular weight (25 KDa).

FIG. 24 is a graph which shows a comparison between mice (dots) vaccinated with the P. pastoris-derived SARS-CoV-2-RBD-SpyVLPs (‘CVD30’) and those vaccinated with the material produced in mammalian cells (‘Expi293’ and ‘ExpiCHO’) The graph shows comparable immunogenicity of the material secreted from Pichia as iLOV-EK-SpyTag-SARS-CoV-2-RBD and subsequently purified (CVD030_F1 and CVD030_F2).

FIG. 25 shows a schematic of the pDcuB::iLOV plasmids where metabolite-responsive regulatory regions were used to determine microbial host productivity. Plasmid pAMK051 (‘A’) harbors a copy of iLOV under the control of the C4-carboxylates-responsive promoter pDcuB; in addition to the pDcuB::iLOV cassette, pAMK052 (‘B’) harbors one copy of the DcuS/DcuR operon under the control of the DcuS promoter, and, pAMK053 (‘C’) harbors a synthetic iLOV-DcuS-DcuR operon under the control of pDcuB.

FIG. 26 is an exemplary plasmid sequence of pAMK051<SEQ. ID NO. 12>.

FIG. 27 is an exemplary plasmid sequence of pAMK052<-SEQ. ID NO. 13>.

FIG. 28 is an exemplary plasmid sequence of pAMK053<SEQ. ID. NO. 14>.

FIG. 29 shows an increase in iLOV fluorescence following exogenous succinate addition to wildtype E. coli ATCC8739 harbouring the different metabolite-responsive regulatory region plasmids (A, B or C) under aerobic conditions.

FIG. 30 shows iLOV fluorescence (black bars) closely matches in vivo succinate levels (closed triangles) produced under anaerobic conditions by an E. coli overproducer, with strain carrying plasmid ‘C’ showing highest fluorescence levels at comparable succinate concentrations. The genetic background empty vector control strain (‘Control’) is shown as a reference,

FIG. 31 shows validation of plasmid biosensor pAMK052 (B) in E. coli strain BW25113 with genotype AldhA ApflB AptsG capable of secreting succinate under anaerobic conditions. Recombinant strain carrying plasmid B shows higher fluorescence relative to the empty vector control strain (‘EV’) at comparable succinate yield. Wild type E. coli strain BW25113 (‘WT’) shows low succinate production and comparable fluorescence to EV.

### DETAILED DESCRIPTION OF A PREFERRED EMBODIMENT

While this invention is susceptible of embodiments in many different forms, there is shown in the appended drawings and will herein be described in detail at least one preferred embodiment of the invention with the understanding that the present disclosure is to be considered as an exemplification of the principles of the invention and is not intended to limit the broad aspect of the invention to any of the specific embodiments illustrated.

Applicant has devised and tested the use of the iLOV fluorescent protein as a general means to characterize diverse P. pastoris transformants and identify those with the highest productivity and stability such that high productivity is sustained over long periods of culture and production of the fused iLOV-target (i.e. genetic stability). iLOV is a small (15 kDa) protein that can be used much like the more widely used GFP but has the advantages of being smaller (so less burdensome to transformants for protein synthesis resources, less intrusive on the fusion partner than GFP and less stoically hindersome to the enzyme used to subsequently cleave it from the fusion partner), can fluoresce in the absence of oxygen (unlike GFP), is stable at high temperatures and across a wide range of pH, and can be easily secreted from P. pastoris (thereby also detecting transformants that secrete the fused target most effectively). While P. pastoris is the most preferred host for the disclosed methods, other hosts including E. coli, Saccharomyces cerevisiae, Bacillus spp, Pseudomonas putida, Chinese Hamster Ovary (CHO) and Human Embryonic Kidney (HEK) may also be suitable for use.

Critically, the present invention includes an enterokinase cleavage site in the protein linker between iLOV and the target protein to allow scarless removal of iLOV and all linker sequences following production in P. pastoris and recovery from the culture. Also critically, the present invention discloses another constructed P. pastoris strain that produces enterokinase in high levels, thereby reducing its cost of use by over 1,000 fold, making it commercially viable. The combination of the iLOV and low cost enterokinase is very advantageous to the time saving and quality of the recombinant P. pastoris isolated in the first example discussed below.

The present invention has multiple uses and benefits over methods used in the art to identify the most industrially viable P. pastoris transformants and potentially other biological production systems.

A further benefit, resulting from the enormous throughput of FACS (Fluorescence Activated Cell Sorter) based screening of fluorescing P. pastoris transformants, is the ability to eliminate a selectable antibiotic resistance marker from the DNA fragment that will express the heterologous target protein of interest. Normally, a gene that encodes a selectable matter conferring antibiotic resistance (eg. resistance to zeocin or kanamycin antibiotics) upon transformed P. pastoris cells must be included and co-expressed from the DNA fragment being introduced in order to identify those P. pastoris cells which have taken up, integrated the heterologous DNA fragment and express the protein target. This is a quite separate and prior requirement to identifying those transformants which have integrated the DNA in the most productive and stable construct or configuration from which to express the heterologous protein target. Of many millions of P. pastoris cells exposed to a transforming DNA fragment, typically only a few thousand will take up and integrate the DNA fragment within the genome to express the encoded genes, To identify these transformants, while removing the much larger number of untransformed cells, requires a level of screening that normally can only be practically achieved using a selectable antibiotic resistance marker that only allows transformed cells to survive. The transformants are then subsequently screened for performance and stability. However, the gene expressing the antibiotic resistance marker also remains in the chromosome in equal copy number to those of the gene encoding the protein of interest. Therefore, P. pastoris transformants that contain many integrated copies of the gene of interest also contain many copies of the antibiotic resistance marker. Because the expression of the antibiotic resistance marker is typically unregulated (constitutive) rather than being induced at a specific time, as is typically the case for the target protein, the expression of the marker often imposes a continual burden upon the host cell. This burden is often manifest by slower growth of the P. pastoris host but, mote importantly, it creates a selective pressure upon the cell to minimize this burden by removing (deleting) the DNA encoding the antibiotic resistance marker. The most likely means by which the DNA encoding the antibiotic resistance marker is removed is through homologous recombination between contiguous copies of the gene encoding the antibiotic resistance marker integrated within the P. pastoris genome. Since contiguous integrated copies of the gene encoding the antibiotic resistance matter are accompanied (interspersed) by copies of the target gene, deletion of the former inevitably results in corresponding deletion of the latter. Accordingly, the presence of a gene that encodes a constitutively expressed antibiotic resistance matter provides a direct selection for deletion of one or more copies of the target, both reducing productivity and introducing highly undesirable genetic instability of the engineered P. pastoris production strain. The very high throughput (6000 cells/second) of FACS based detection of transformants expressing an inducible (or constitutive) fluorescent marker overcomes the normal practical limitation of otherwise screening for P. pastoris transformants, eliminating tin: need to include a gene encoding an antibiotic resistance marker, offering a major benefit to the genetic stability and consistent productivity of a P. pastoris strain engineered to produce a target protein from multiple integrated copies of a heterologous gene sequence.

A further application of the iLOV protein fusion is to more easily and rapidly assess criteria that affect the efficiency with which the same heterologous protein can be expressed in recombinant P. pastoris strains. These criteria include choice of regulatory region (promoter, ribosome binding site, transcription terminator) or the choice of gene codon composition and context. Also, the iLOV protein fusion can more easily and rapidly compare and monitor the stability of integrated genetic constructs derived from mixtures of genes of differing regulation and/or codon composition and context that all encode the same heterologous protein.

A further application of the scarless removal of the iLOV fluorescent marker by enterokinase is the ability to also include on the removable protein fragment, sequences of protein that facilitate the purification of the fusion protein prior to their removal. Such sequences could for example include amino acids that increase the acidity, basicity or hydrophobicity of the protein fusion such that they are more readily separated by chromatographic methods well known in the art.

### EXAMPLES

### Example 1

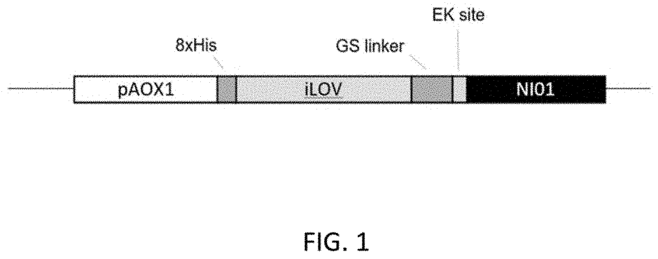

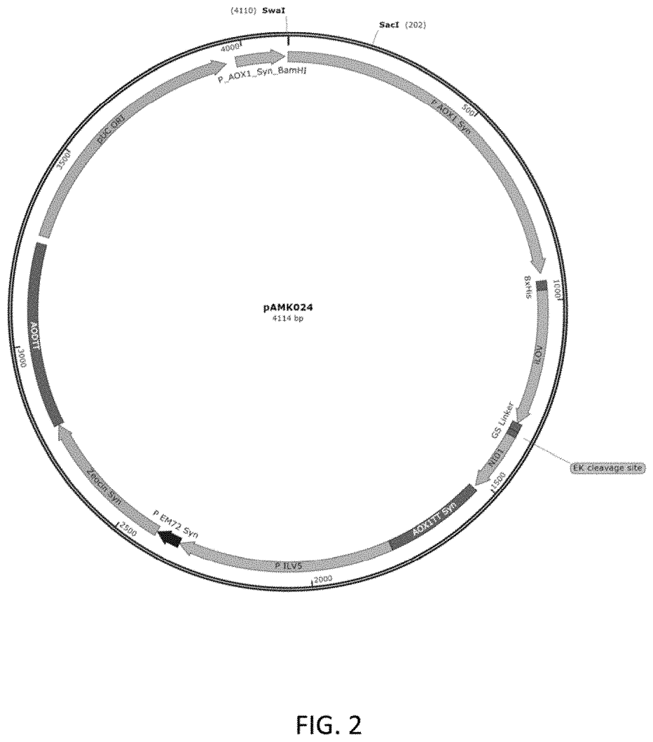

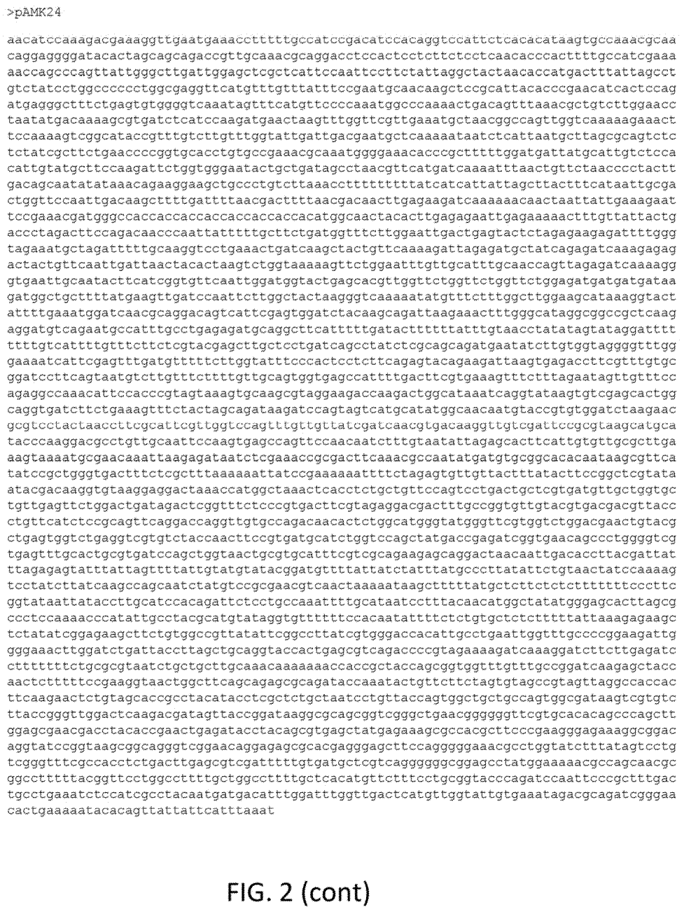

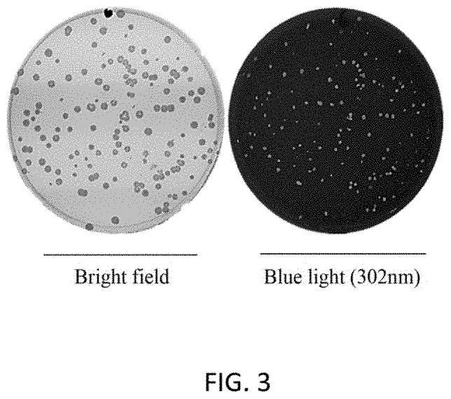

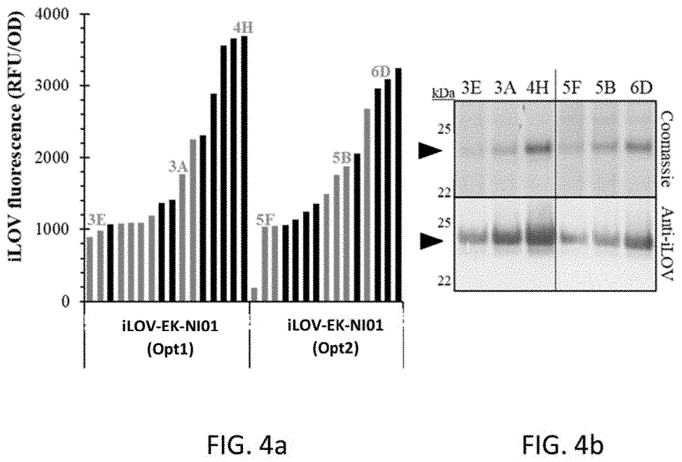

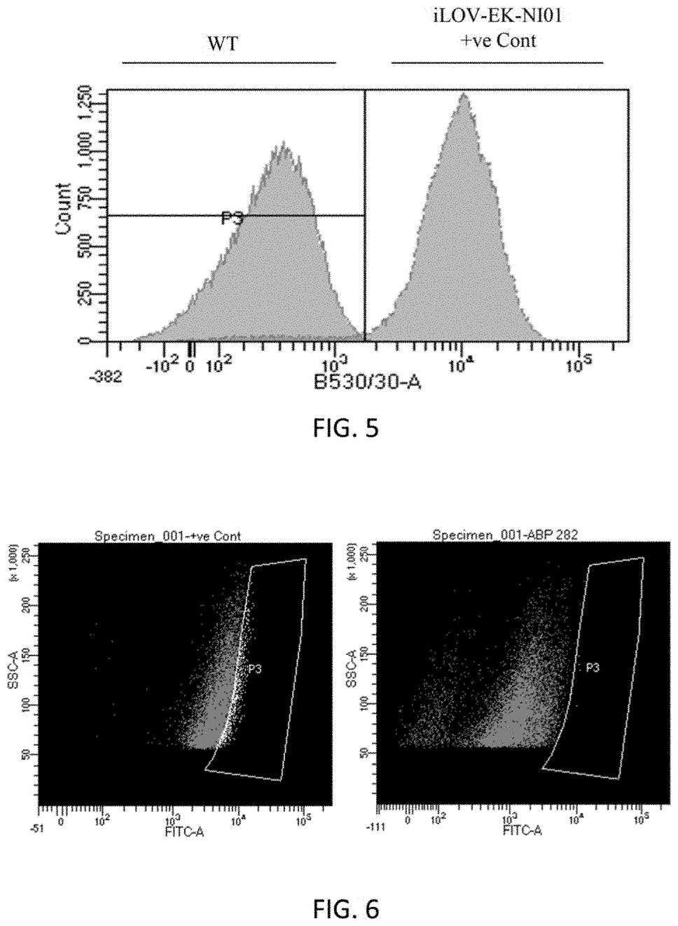

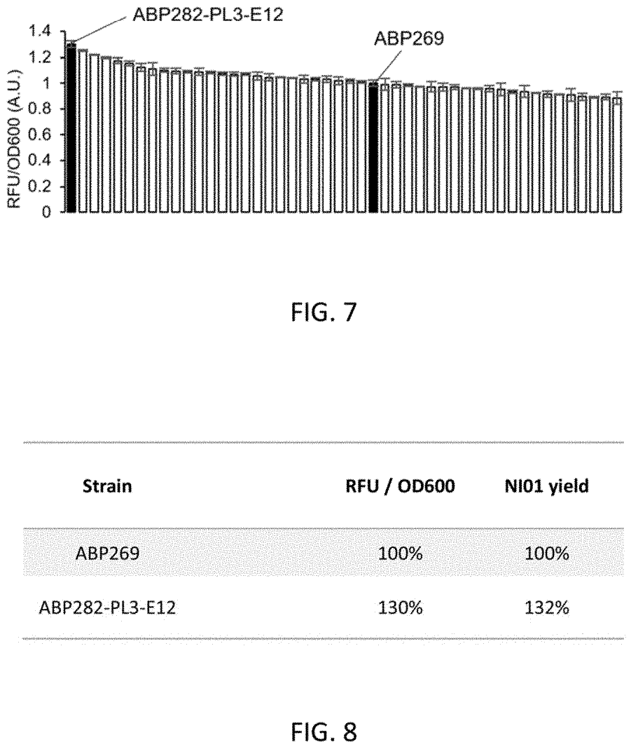

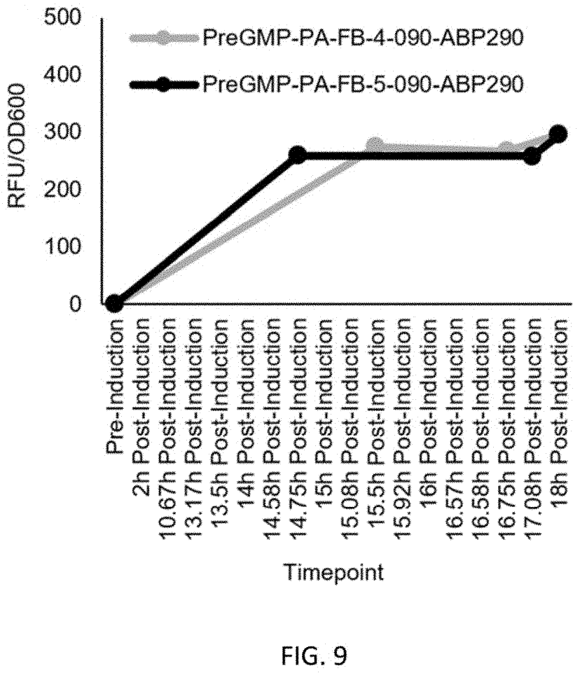

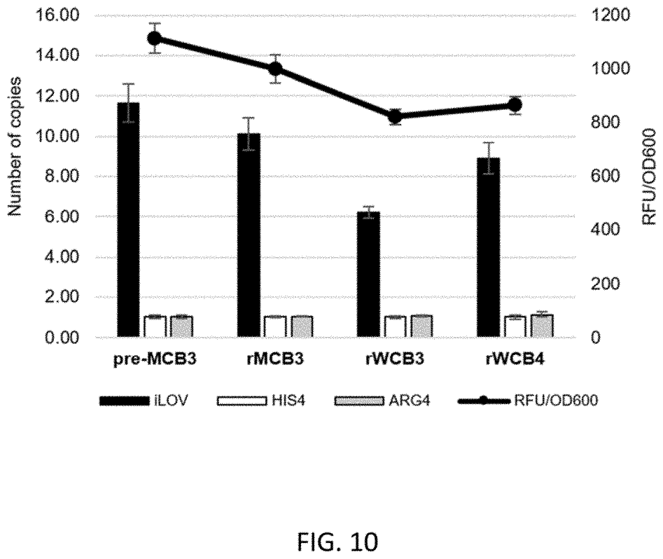

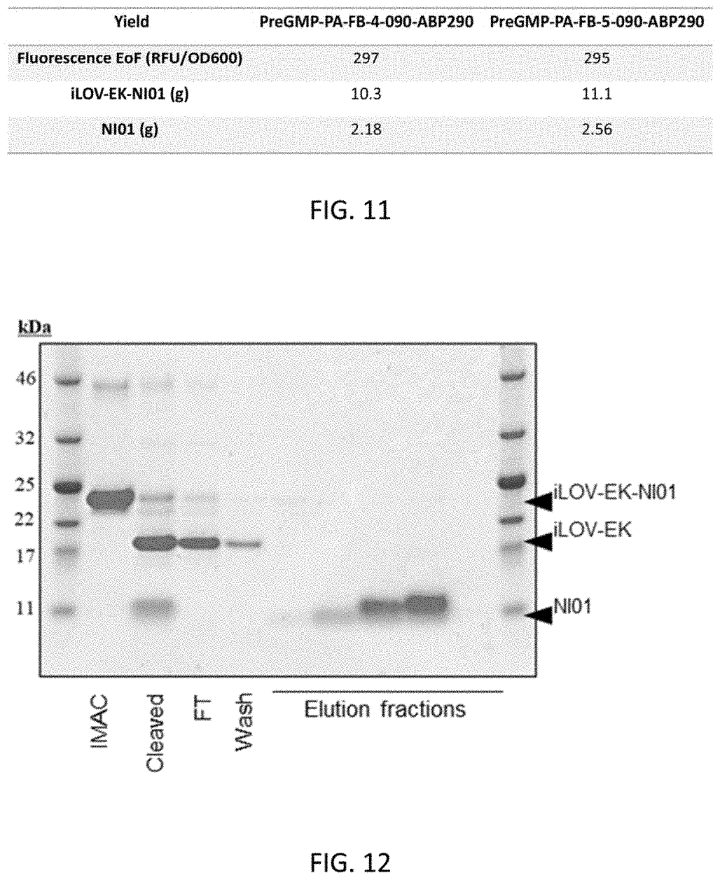

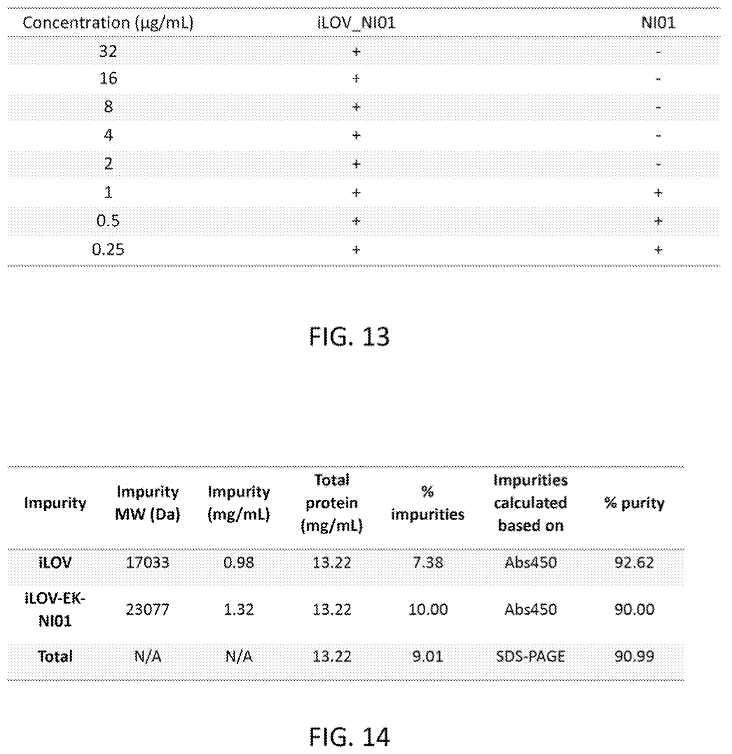

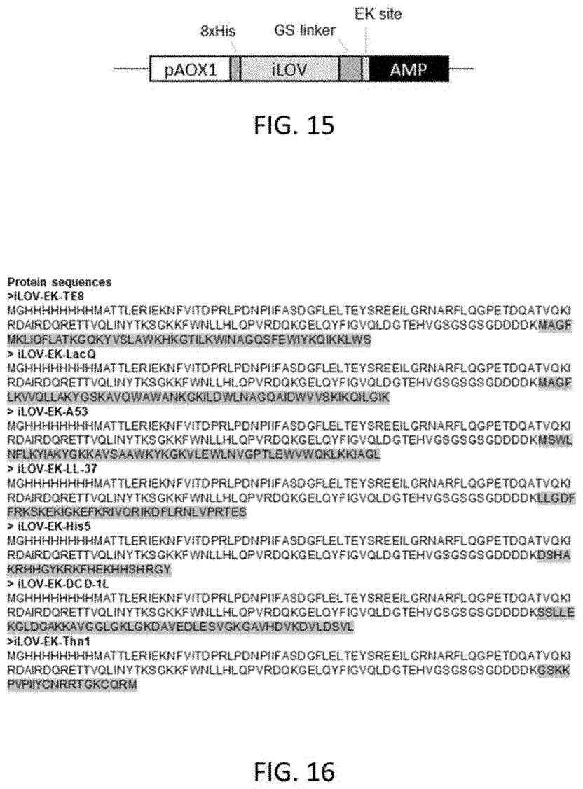

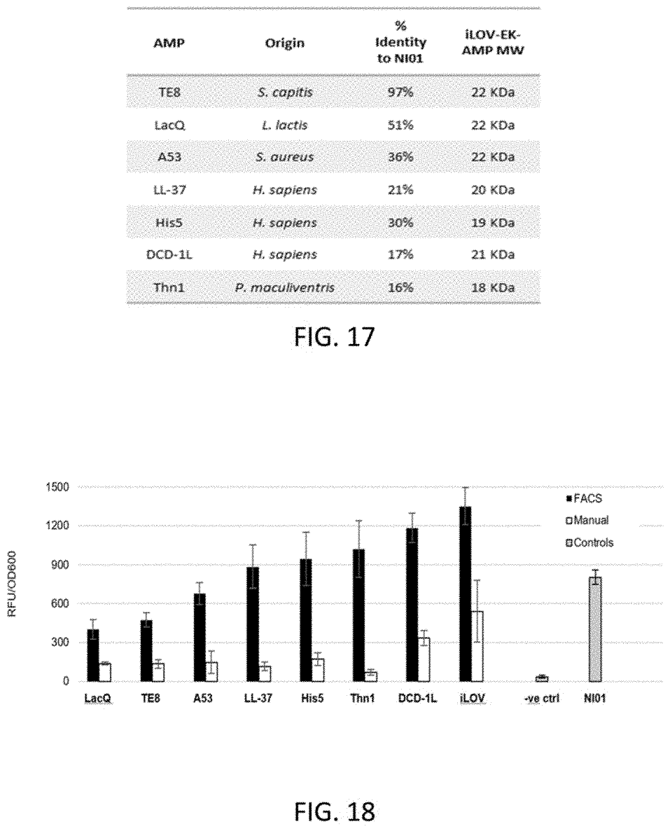

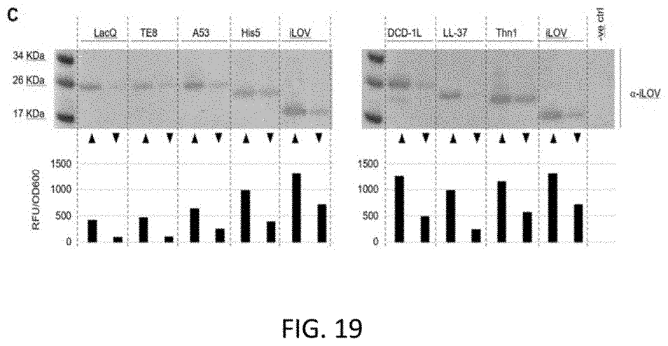

With reference to FIGS. 1-19, details of a first example are illustrated. The disclosed first example uses P. pastoris to produce a protein comprising iLOV, an enterokinase cleavage site and a protein linker, fused to a protein named epidermicin-N101, a 51 amino acid antimicrobial protein that is highly cytotoxic to microbial species including “superbug” bacteria such as methicillin resistant Staphylococcus aureus (MRSA). The epidermicin-N101 was discovered by researchers at Plymouth University and is of high biomedical relevance. But it could only be produced in tiny quantities by the original researchers as it was highly toxic to recombinant microbes used to produce it. Applicant developed iLOV-EK-N101 protein fusion in P. pastoris, achieving greater than 1,000-fold increased production over non-fused recombinants, which is at commercially relevant levels. By “masking” much of the toxicity of N101 to P. pastoris, the iLOV fusion helps to identify the most stable and productive transformants.

For example, hundreds of individual P. pastoris transformants were screened by simply illuminating them on petri plates with blue light and observing fluorescence, rather than having to assay N101 activity which would have allowed perhaps 20-30 transformants to be screened in a much longer timeframe. A FACS machine was then applied which within an hour could screen millions P. pastoris transformants. Levels of fluorescence detected from individual transformants closely mirrored product yield, permitting identification of productivity improvements and accumulation of the protein throughout fermentation.

Further, genetic stability of the recombinant strain, reproducibility of the upstream/downstream processing and QC of the recombinant material can all be monitored via iLOV fluorescence. iLOV could be used as a conditional precipitant upon removal of salt, allowing isolation of the soluble fraction, conditional precipitation by salt removal, separation via filtration, and then re-solubilization of relatively pure protein by exposure to a higher salt concentration. The advantage of this method is that the target is initially soluble, thus removing the need for urea/guanadine:HCL solubilization of the precipitated target protein. Thus, the gain in throughput and time saving is enormous.

The method can be applied to alternative antimicrobial peptide (AMP) homologue sequences with low identity to N101; these include, but are not restricted to, Thanatin, Dermicidin, Lacticin Q, Aurecin A53, Histatin 5, TE8 and LL-37.

### Example 2

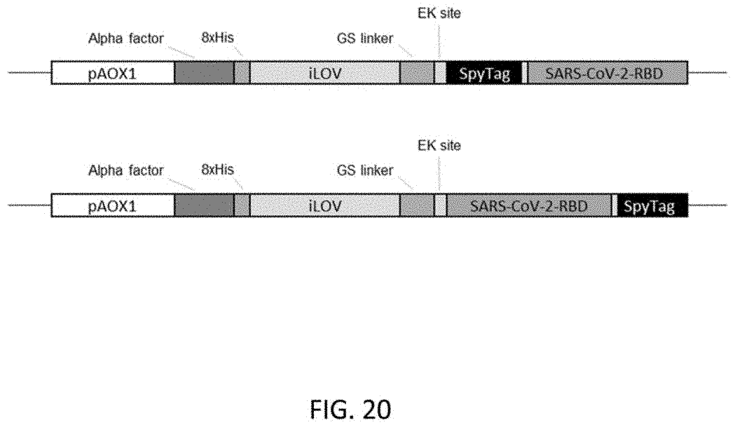

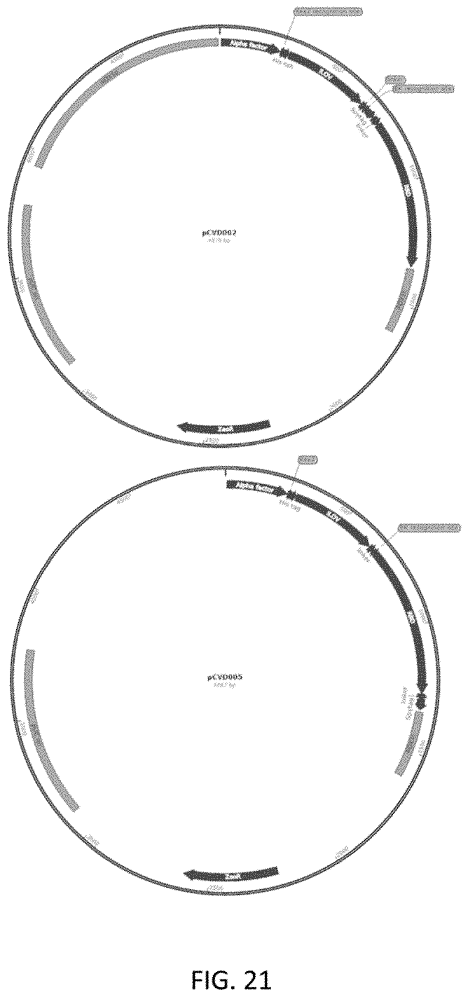

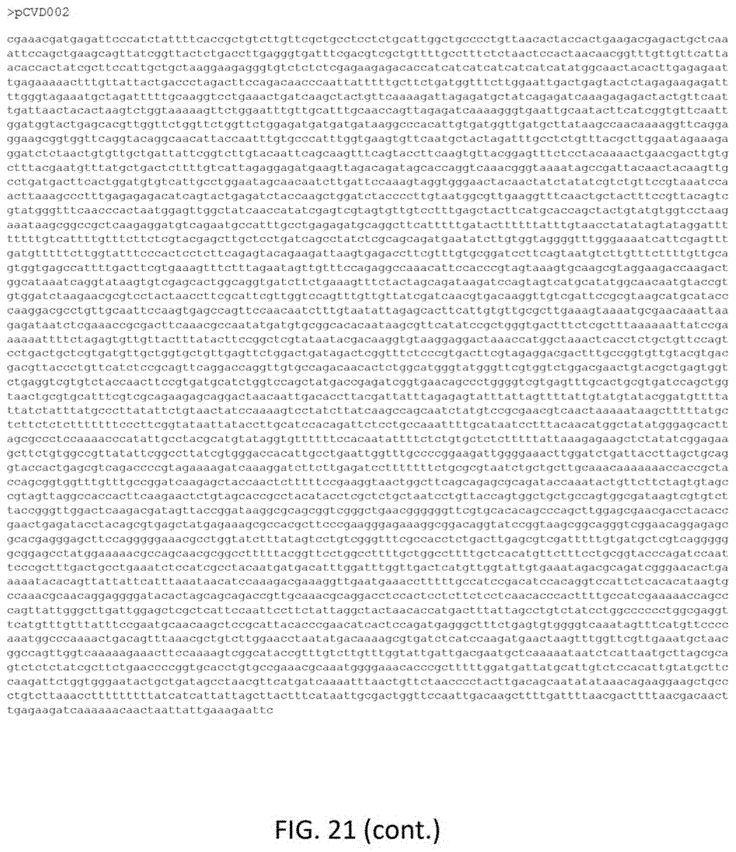

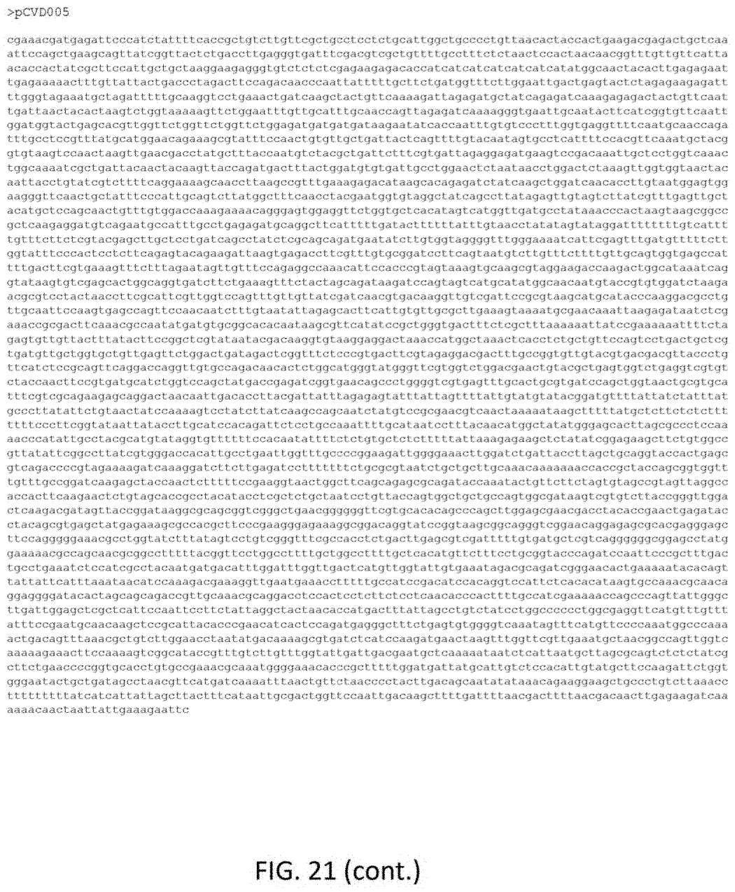

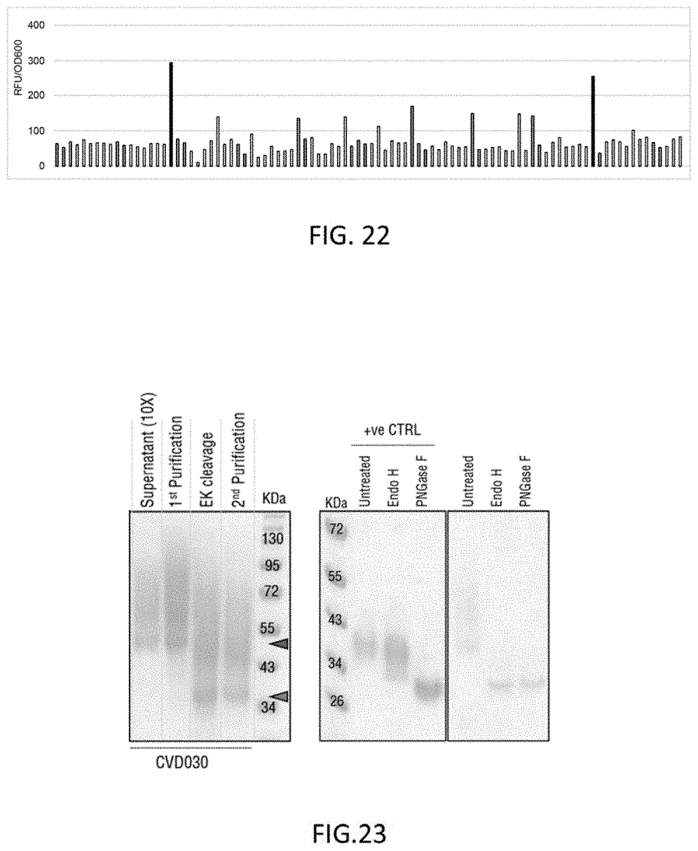

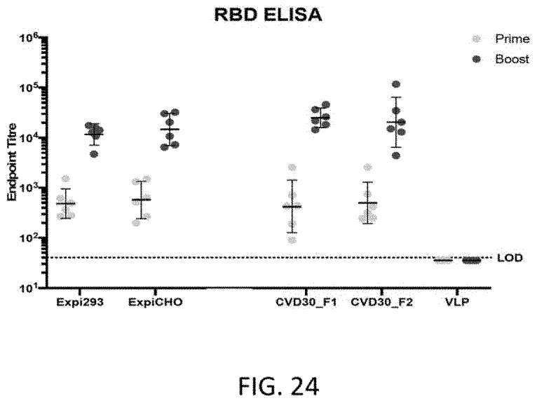

With reference to FIGS. 20-24, a second example can be more readily understood. In the second example, recombinant P. pastoris is used to produce an iLOV-enterokinase linker fused to the 199 amino acid Receptor Binding Domain (RBD) of foe SARS-CoV-2 viral spike protein. The RBD attaches to a virus like particle (VLP). A VLP is a self-assembling structure formed by a monomer such as “mi3”. Mi3 is flanked by short amino acid linkers called “SpyCatcher”. The monomer-SpyCatcher protein is expressed in E. coli and spontaneously assembles into a soccer ball-like structure from which the SpyCatcher tag protrudes. The RBD is attached to a “SpyTag” peptide that facilitates iso-peptide bond formation to a corresponding “SpyCatcher” domain on a VLP vaccine delivery vehicle.

Applicants thereby developed a SARS-CoV-2 VLP based protein subunit vaccine using SARS-CoV-2 RBD produced in engineered P. pastoris strains and secreted from foe P. pastoris as iLOV-EK-SpyTag-RBD fusions. Enterokinase cleavage of the material secreted from P. pastoris cells allowed recovery and purification of SARS-CoV-2 SpyTag-RBD which when conjugated to mi3 SpyCatcher-VLP showed comparable immunogenicity to RBD recombinantly produced in mammalian cell lines in vitro and mice, potentially overcoming the cost, scalability and productivity issues associated with current mammalian cell-based vaccine production. A FACS machine or microfluidic droplet platform can be used for preliminary selection of high-expressing clones. Measuring fluorescence of iLOV-EK-RBD fusions in P. pastoris culture media using a plate reader allows detection of optimally productive recombinant strains.

### Example 3

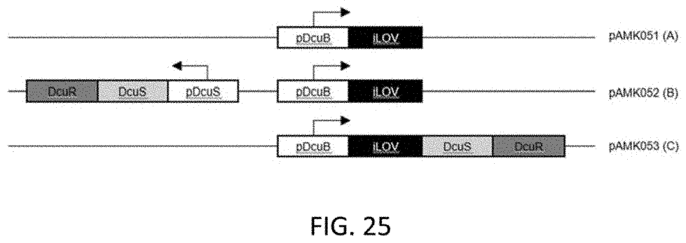

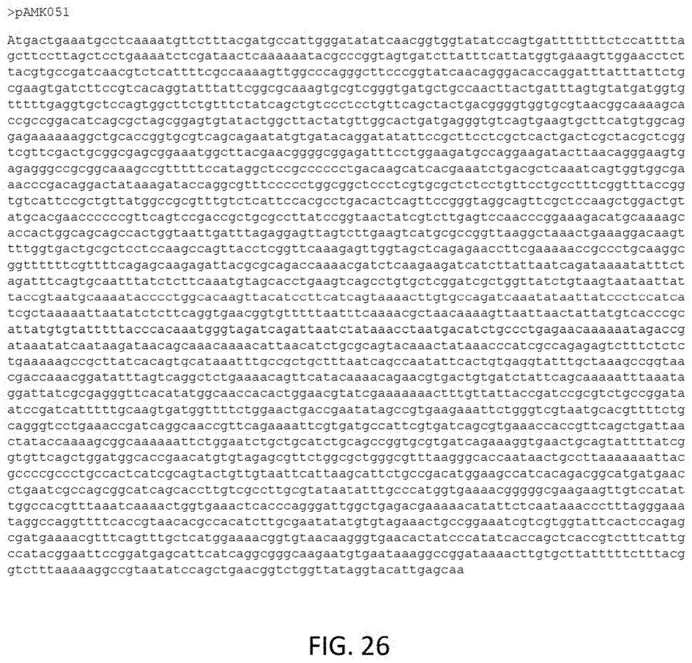

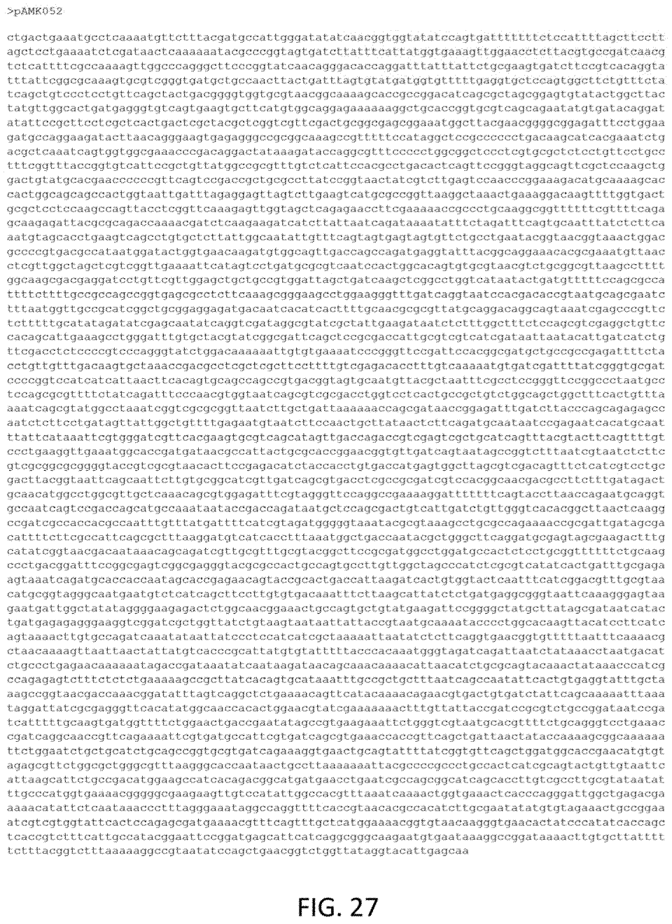

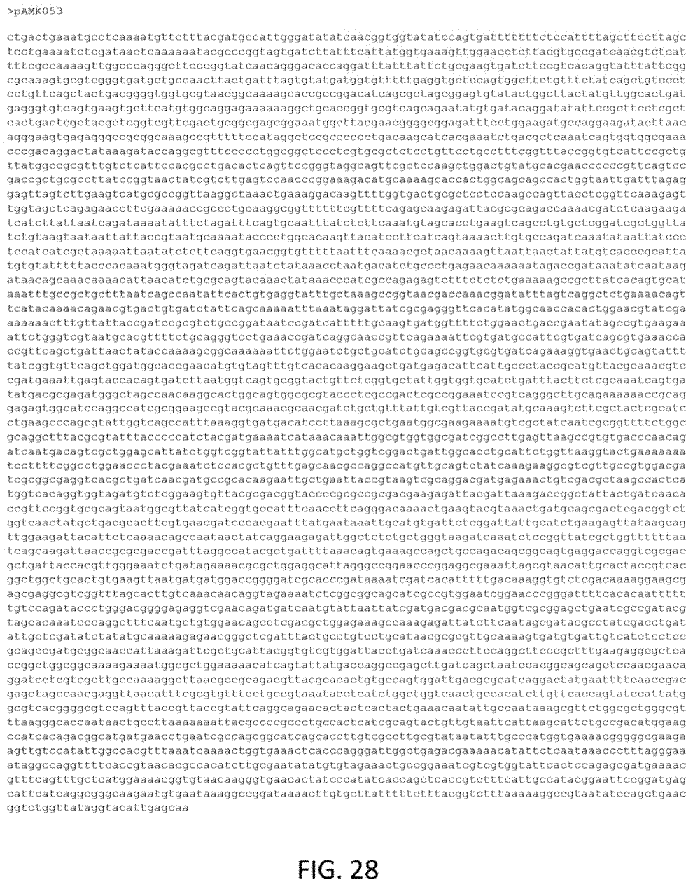

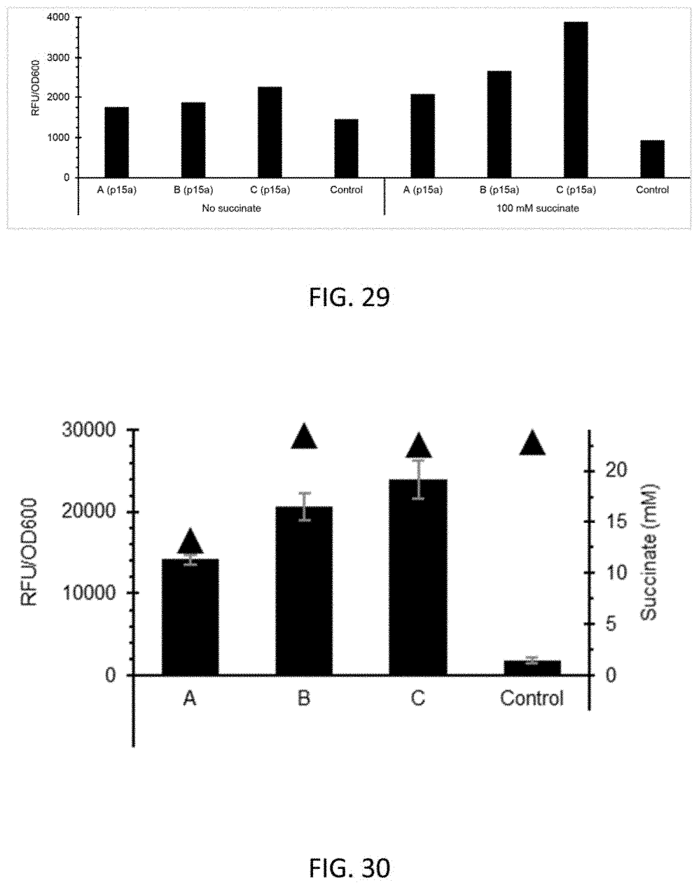

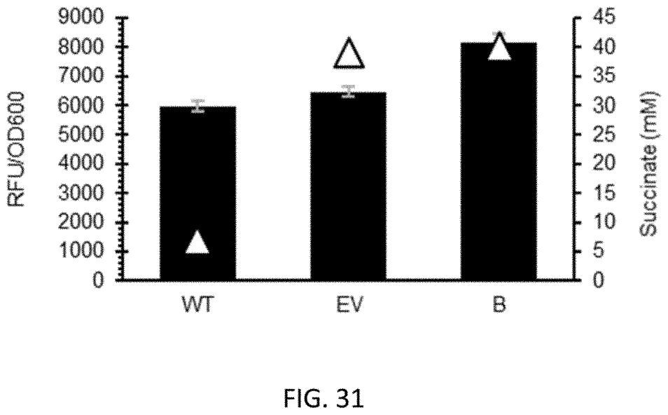

Finally, a third example is more readily understood with reference to the images of FIGS. 25-31. In the third example, identification of the most effective metabolite-responsive DNA regulatory regions based on fluorescence outputs is allowed by placing the reporter iLOV under the control of such metabolite-responsive DNA regulatory regions in presence of the metabolite. Similarly, in vivo metabolite productivity of individual microbial hosts that have been transformed with a metabolite-responsive DNA regulatory region driving the expression of iLOV can be estimated, thus removing the requirement for time consuming offline biochemical/analytical assays. The system is adaptable to both aerobic and anaerobic processes.

Succinate is an added-value chemical produced by E. coli at later growth stages under anaerobic conditions, which prevent the use of oxygen-dependent fluorescent reporters such as GFP and related proteins. Increased succinate production is known to regulate gene expression via the two-component DCuS/R feedback system. This system relies on phosphorylation of the response regulator DcuR by the transmembrane domain DcuS in the presence of C4-carboxylates, such as fumarate and succinate; phosphorylated DcuR activates transcription of the €4-dicarboxylate exporter DcuB. Applicant developed biosensor plasmids where iLOV expression is placed under the control of the DcuB regulatory region and showed an increase in specific fluorescence when E. coli strains are either supplied with exogenous succinate aerobically or allowed to produce succinate in vivo under anaerobic conditions. High-throughput identification of superior C4-carboxylates-producing E. coli clones under anaerobic conditions can be envisaged using this method as a biosensor. Altered regulatory regions (e.g. microbial native metabolite-responsive promoters) could be used to drive inducible or constitutive expression of a recombinant protein of interest (POI) in E. coli, where the iLOV reporter sequence exemplified here could be replaced by iLOV-EK-POI fusions. The system allows real-time monitoring of metabolite and/or protein production in E. coli, thus increasing throughput, optimization of upstream processing conditions and screening of regulatory regions of interest that are responsive to specific metabolites.

### Material and Methods

Transformation of P. pastoris and Libraries Generation

P. pastoris library construction was performed using strain BSYBG11 Mut^{s} (BioGrammatics, bisy e.U.) as genetic background. For preparation of electrocompetent cells, 100 mL culture of P. pastoris BSYBG11 was grown in Yeast Extract-Peptone-Dextrose (YPD; 2% w/v glucose) at 30° C., 250 in a baffled Erlenmeyer flask until an OD600˜1.0 was reached. The culture was then cooled on ice for 30 min before centrifugation at 1600 rpm at 4° C. for 10 minutes. Supernatant was discarded, and the cells resuspended in 9 mL ice-cold BEDS solution (10 mM Bicine-NaOH, 3% v/v ethylene glycol, 5% v/v dimethyl sulphide, 1 M D-sorbitol) with addition of 1 mL DTT (100 mM final concentration). Cells were then incubated at 30° C. with 200 rpm shaking for 5 minutes before centrifugation at 1600 rpm, 4° C. for 5 minutes; supernatant was discarded and cell pellet was resuspended in 0.5 mL BEDS solution without DTT. Competent cells aliquots (40 μL) were kept on ice and transformation was undertaken within 2 hours. Plasmids were linearized by digestion with SacI or SwaI (NEB) for 1 hour at 37° C. and subsequently purified using the Monarch® PCR & DNA Cleanup Kit (NEB) prior to transformation.

Approximately 1 μg linear DNA was added to 40 μL electrocompetent cells and electroporation performed at 1.5 kV, 200 fit, 25 μF in a 2 mm electroporation cuvette using a Bio-Rad GenePulser electroporator. Following electroporation, cells were resuspended in 1 ml Yeast Extract-Peptone-Dextrose-Sorbitol (YPDS; 0.5M D-Sorbitol) and allowed to recover at 30° C., 250 rpm for 3 hours. Transformants were selected on YPDS plates (1.2% w/v agar) containing 100 μg/mL, 250 μg/mL or 500 μg/ml. Zeocin following incubation for 2 days at 30° C.

Solid- and Liquid-Phase Screening of iLOV-EK-ABP Transformants

For solid-phase screening, sterilized Whatman filter papers were placed onto YPDS+Zeocin transformation plates and pressed onto colonies using a cell spreader. The paper was removed and placed onto a minimal media induction agar plate (Yeast Nitrogen Base, 1% v/v methanol). Plates were scaled to avoid evaporation of methanol and incubated at 30° C. for two days before filter papers were removed and additional 200 μL of 50% v/v methanol were added to the lid of the upside-down plates to keep induction at 30° C. for a further 24 hours. Original transformation plates were likewise incubated to allow colonies to re-form. Pictures of induction plates were recorded under UV-light and of transformation plates under white light. The images were overlaid to track fluorescence of individual colonies and identify high-expressing clones.

For liquid-phase screening, selected colonics were picked from the original transformation plates and resuspended in 1.75 mL BMGY in 24-deepwell plates (square wells). Plates were sealed with a sterile breathable film and incubated at 30° C., 250 rpm with 75% humidity for 48 hours. 100 μL of culture was removed to generate glycerol stocks before plates were centrifuged at 3000 rpm for 10 minutes.

For induction of protein expression, supernatant was discarded, and the biomass resuspended in 1.75 mL induction media (BMMY, 0.5% v/v methanol) per well. After 24 hours incubation at 3° C., 250 rpm with 75% humidity, 200 μL of culture was transferred to a 96-well black-walled clear-F-bottom plate (Costar) and fluorescence recorded at 450 nm excitation, 500 nm emission. Fluorescence values were typically normalised by OD600.

For SDS-PAGE analysis, culture samples were normalised to OD600=5.0 and lysed by addition of YeastBuster™ (Merck). SDS-PAGE samples were prepared with 1×Bolt™ LDS Sample buffer (Thermo Fisher) and 0.0% (w/v) DTT, denatured at 100° C. for 5 minutes. Pre-cast Bolt™ BisTris 4-12% polyacrylamide 1 mm thick gels (Thermo Fisher) were used for analysis. As standard, 10 μL of sample were loaded onto the gel and electrophoresis performed in 1× MFS buffer (Thermo Fisher) at 120V for 55 minutes.

To indicate molecular weights, 2.5 μL of Color Prestained Protein Standard, Broad Range (NEB) were included on each gel. Western blot analyses were performed following protein transfer from the polyacrylamide gels onto PVDF membranes using the iBlot 2 Dry Blotting System (Thermo Fisher). Gels were placed onto the blotting membrane in a Transfer Stack and this was reassembled and placed in the Transfer Device. Standard program P0 was run (20 V for 1 minute, 23 V for 4 minutes, 25 V for 2 minutes). Membranes were incubated for 1 hour in 20 mL phosphate buffered saline with 0.2% (v/v) Tween-20 (PBST) buffer with 5% (w/v) milk powder (Asda) in a 50 mL falcon tube on a tube roller. Primary antibodies (rabbit-anti_phiLOV, Prof. John Christie Univ. of Glasgow) were added at 1:2500 dilution to milk-PBST and the membranes incubated for 1 hour at room temperature. Three washes with 15 mL PBST for 5 min each were performed. Secondary antibodies (goat-anti rabbit HRP conjugate. Fisher Scientific) were added using a 1:10000 dilution to milk-PBST and allowed to incubate for 1 hour at room temperature. Following three washes, immunodetection was performed using DAB in stable peroxide buffer (Thermo Fisher) for approximately 10 minutes until clear signals could be observed. Membranes were rinsed with excess water and dried before scanning.

PreGMP fermentation runs of recombinant P. pastoris strains were conducted using an optimised, defined minimal media and process where agitation, pH, temperature, dissolved oxygen and exhaust gases are monitored throughout. Fluorescence levels per OD600 were assessed at specific timepoints and at the end of fermentation (EoF).

### Flow Cytometry and FACS

For high-throughput analysts and sorting of recombinant P. pastoris strains expressing high levels of iLOV-EK-N101, iLOV-EK-ABP or iLOV-EK-SARS-CoV-2-RBD, colonics selected on YPDS+Zeocin transformation plates were washed using 7 mL of BMGY and this mixed transformant population used to inoculate 50 mL BMGY in 250 mL shake flasks at an initial OD600=0.1. Cultures were allowed to grow at 30° C., 250 rpm, for three days and subsequently cell biomass was separated by centrifugation and gene expression induced by replacement of the BMGY growth media with BMMY induction media containing 0.5% (v/v) methanol.

After 24, 48 or 72 hours from induction, cell populations were normalised to OD600=0.75 using BMMY as a diluent. Flow cytometry and FACS were carried out on a BD LSR Fortessa and a BD FACS Aria IIIu 4-laser/11 detector Cell Sorter respectively (488 nm excitation laser; FITC emission filter). High-expressing clones were sorted onto 96-well plates containing 100 μL of YPD+Carbenicillin (100 □g/mL) and allowed to grow at 30° C. for two days.

Confirmation of performance was performed using liquid-phase screening in 24- or 96-deep well plates. For flask experiments, stable P. pastoris transformants were grown in 50 or 500 mL of antibiotic-free BMGY media for 48-72 hours, (ell biomass was then centrifuged and resuspended in induction media BMMY containing 0.5% v/v methanol.

For long term storage of cultures, glycerol stocks were generated from overnight cultures inoculated with a single colony, supplemented to 10% (v/v) glycerol and stored at −80° C.

Genomic DNA (gDNA) was extracted from stable P. pastoris integrants grown in YPD for 24 h using the YeaStar Genomic DNA kit (Zymo Research) following the manufacturer's instructions. The incubation step was increased to 1 hour and 30 min. Samples were eluted in 40 μl nuclease-free water. Copy number analysis of strains expressing iLOV-EK-N101 fusions was performed using iLOV-specific primers due to differences in the N101 codon-optimisation tested; three housekeeping genes (HIS4 and ARG4) were used as internal loading control for normalisation following the ΔΔCq method. Forward and reverse primer sequences are as follows:

ILOV
(ACCCTAGACTTCCAGACAACCC/
GCTTGATCAGTTTCAGGACCTTGC)
HIS4
(TTTGACTACTGACCGCCCCG/
ACGAGTACACCAGGCCCAAC)
ARG4
(GCAGAGTGGGCAGAAGGGAA/
ACTCACCCAAGCGACGTTCA)

Reaction mix was prepared for each pair of primers in a 10 μl final volume using the 5 μl PowerUp SYBR Green Master Mix 2× (Thermo Fisher), 0.5 μl primer forward 10 μM, 0.5 μl primer reverse 10 μM and 4 μl of a 100-fold dilution of the 1 ng/μl gDNA sample. Thermocycler conditions used were; 50° C. for 2 min, 95° C. for 2 min followed by 40 cycles (95° C. for 15 sec, 60° C. for 1 min) and a melting curve analysis.

### Purification of N101

P. pastoris recombinants expressing iLOV-EK-N101 were lysed overnight using a proprietary lysis buffer. The cell lysate was subsequently clarified by tangential flow filtration resulting in approximately 4× dilution of the starting material with 50 mM TrisHCl, pH 8.0, 150 mM NaCl (Buffer A). Zinc IMAC purification was performed on an AKTA Pure 150 (OH Healthcare) following recommended protocol.

Briefly, a HiPrep IMAC FF 16/10 (GE Healthcare) column was prepared washing with 10 column volumes (CV) diH2O followed by 15 ml, of 100 mM ZnCl2 pH 3.0 to load the zinc ions onto the resin. Column was further equilibrated to remove all unbound zinc with 10 CV ddH2O, H) CV Buffer A, 5 CV Buffer B (50 mM TrisHCl, pH 8.0, 150 mM NaCl, 500 mM imidazole) and finally 5 CV Buffer A. 400 mL of cell lysate were loaded onto the column and resin was washed with Buffer A until the flow through reached a stable (JV-absorbance at 280 nm.

Concentration of Buffer B was increased to 100% over 7.5 CV followed by 2 CV of 100% Buffer B. 10 mL fractions were collected and analysed by SDS-PAGE for iLOV-EK-N01. Fractions containing iLOV-EK-N101 were pooled and stored at 4° C. overnight. Enterokinase cleavage was performed following incubation for 3 hours with 0.2% (w/w) Ingenza recombinant enterokinase (rEK) enzyme at 30° C. N101 was purified by CIEX following cleavage from iLOV-EK using an ÄKTA Pure 150 (GE Healthcare) with a HiTrap SP FF column. The column was equilibrated with 50 mM CHES pH 9.4 (Buffer A) and the reaction mixture containing iLOV, N101 and rEK proteins was loaded onto the column. The resin was washed with Buffer A until a stable UV-absorbance at 280 nm was observed. N101 was eluted by gradual increase of 50 mM CUES, pH 9.4, 1 M NaCl (Buffer B) to 100% of Buffer B over 20 CV. 5 mL fractions were collected. N101 yield and protein concentrations were measured by absorbance at 280 nm in a plate reader using a UV star 96-well plate, half area. A 40-fold dilution of the pre-concentrated material was made.

A light scattering correction was used by measuring absorbance at wavelengths 320, 325, 330, 335, 340, 345 and 350 nm. The logarithm of the observed absorbance was then plotted against the logarithm of the wavelengths to create a standard curve by linear regression. The curve was extrapolated to determine the logarithm of the absorbance at 280 nm. The antilogarithm of this value attributed to light scattering—was then subtracted from tire total absorbance measured at 280 nm to obtain the value of the protein in solution. Extinction coefficient for N101, 4.133 ml/mg, was used to calculate the peptide concentration by the Beer-Lambert law.

### Determination of Minimum Inhibitory Concentration (MIC)

Micrococcus luteus was used as the test organism to determine potency of the purified N101. This organism was streaked onto an LB plate from a glycerol stock and incubated at 37° C. for 48 h until isolated colonies were observed. A 0.85% (w/v) NaCl solution was prepared and filter sterilised. 3 ml of this saline solution were transferred to a 15-ml tube and 3-4 colonies of M. luteus were resuspended in it to reach OD600 between 0.08 and 0.12, corresponding to a microbial suspension of 1-2×10⁸ CFU/ml. A 150-fold dilution was made in 4.5 ml f Mueller Hinton Broth (MHB) to result in a microbial suspension of approximately 10⁶ CFU/ml.

A 96-well round bottom plate was used to set up the assay. In each well, 50 μl of this suspension was added to 50 μl of N101 dilutions at the appropriate concentration (0.25 to 32 μg/mL) prepared using MHB as a diluent. Plates were sealed with a breathable seal and placed in the static incubator at 37° C. for 24 hours. MIC is determined after visual inspection and defined as the concentration of N101 where no M. luteus growth is observed.

### Quantification of Impurities

Residual iLOV-N101 and iLOV-EK in the purified material were assessed indirectly through quantification of FMN (Ore cofactor of iLOV; 1:1 ratio) at 450 nm using extinction coefficient at 450 nm=12500 M⁻¹ cm⁻¹. Molar concentration calculated by the Beer-Lambert law: A=1cε. Protein concentration (mg/mL) is calculated from molar concentration using the formula mg/mL=M×MW (in Da). Impurities observed as off-target MW bands on SDS-PAGE gel stained with Coomassie Blue and quantified using an imaging software closely match values obtained through absorbance.

### De-glycosylation of SpyTag-SARS-CoV-2-RBD

Recombinant RBD obtained in P. pastoris were dc-glycosylated following treatment with Endo H or PNGase F in non-denaturing conditions (9 mL RBD-containing sample, 1 mL glycobuffer 2 [Endo H] or glycobuffer 3 [PNGasc F] and 75 μL enzyme) following incubation at 37° C., 250 rpm overnight. Decrease in molecular weight following effective de-glycosylation was assessed in western blot using SARS-CoV-2 (2019-nCoV) Spike RBD Antibody (Sino Biological) at a 1:5000 dilution.

### RBD ELISA

SARS-CoV-2-RBD (0.1 μg) obtained from recombinant P. pastoris strains was used to immunise Balb/C and C57BL/6 mice twice using AddaVax as an adjuvant. To detect anti-RBD antibody in the immunised mouse sera, 50 μL purified RBD-6H (amino acids 330 to 532) (2 μg/mL) diluted in PBS was coated on NUNC plates at 4° C. overnight. Plates were then washed with PBS and blocked with 300 μL of 5% skimmed milk in PBS for 1 h at RT. In round-bottom 96-well plates, heat-inactivated mouse sera (starting dilution 1 in 40) was diluted in PBS/0.1% BSA in a 2-fold serial dilution in duplicate. 50 μL of the diluted sera was then transferred to the NUNC plates for 1 h at RT. Plates were then washed with PBS and 50 μL of secondary HRP goat anti-mouse antibody (Dako P0417) diluted 1:800 in PBS/0.1% BSA was added to the wells for 1 h at RT. Plates were washed and developed as described above. Serum RBD-specific antibody response was expressed as endpoint titre (EPT). EPT is defined as the reciprocal of the highest scrum dilution that gives a positive signal (blank+10 SD) determined using a five-parameter logistic equation calculated using GraphPad Prism 8.

Preparation and Transformation of Electrocompetent E. coli Strains

LB no-salt E. coli cultures were inoculated to OD600 0.1 from an overnight culture and grown as described to mid-exponential phase (OD600 0.5-0.7). Cells were chilled on ice for 2 hours before harvesting by centrifugation at 1122×g for 20 min. Harvested cells were washed twice with chilled sterile water followed by one wash in chilled sterile 20% (v/v) glycerol. Cells were then resuspended in chilled sterile 20% (v/v) glycerol, and aliquots of 60 μL prepared in 1.5 mL Eppendorf tubes to be stored at −80° C. until required. For transformation by electroporation, an aliquot of competent cells and up to 5 μL DNA solution was added to a chilled 1 mm electroporation cuvette. An electric current (1.7 k V, 200Ω, 25 μF) was applied using a Bio-Rad GenePulser electroporator. Time constants were between 1.2 and 1.4. The electroporated cells were resuspended in 900 μL SOC media and incubated in a 1.5 mL Eppendorf tube at 37° C., 250 rpm for 1 hour. The cells were appropriately diluted or concentrated according to expected transformation success and streaked onto LB agar plate with the appropriate antibiotic for selection. Plates were incubated overnight at 37° C. to obtain single colony forming units (CFUs).

Succinate Responses in E. coli Strains Expressing iLOV

For experiments using wildtype E. coli ATCC8739 where succinate was exogenously added under aerobic conditions, LB+antibiotic cultures were grown overnight at 37° C., 250 rpm. 25 μL of overnight culture was used to inoculate 5 mL LB in 10 mL glass tubes in the presence of cither 0 or 100 mM succinate. Cells were allowed to grow for 48 hours at 37° C., 250 rpm, after which time 200 μL of culture wen: used to measure OD600 and fluorescence in a 96-well plate with black walls and clear bottom. Measurements were taken in a Tec an Infinite® 200 plate reader with excitation at 450 nm and emission at 510 nm.

For anaerobic experiments on succinate-producing E. coli strains, 200 mL SM aerobic media in a 1 L baffled shake flask was inoculated with 5 mL LB overnight culture and incubated at 37° C., 250 rpm for ˜4 hours to OD600˜4.0. Cells were collected by centrifugation at 1122×g for 12 minutes and supernatant discarded. Cells were resuspended at 7.5% w/v in SM anaerobic media and 1.8 mL of rite resuspension was aliquoted (n=3) into 2 mL Eppendorf tubes. Tubes were scaled with parafilm and incubated at 37° C., 100 rpm. Samples were taken at 24 hours and 48 hours for measuring OD600 and fluorescence intensity at 450 nm excitation. 500 nm emission (FLUOstar Omega Microplate Reader, BMG LABTECH) in a black-walled an F-bottom blade-walled, clear bottom plate (Costar).

For analysis of succinate and glucose content in the cultures, 200 μL of the cell resuspension was boiled for 5 minutes and mixed with 200 μL 200 mM HCl with 0.2% v/v formic acid. This was centrifuged for 5 minutes at 16,000×g and filtered (0.2 μm) prior to HPLC analysis on a REZEX ROA-Organic Acid H+ (8%) column with 0.1% v/v formic acid (0.6 mL/min for 15 min at 65° C.).

### Additional Features

While preferred embodiments of the invention are set forth in the accompanying claims, the following are additional features, advantages and/or alternate embodiments of the disclosed inventive methods.

A target protein produced by a method of providing a DNA sequence encoding a fusion protein, the fusion protein being comprised of a DNA sequence encoding an iLOV protein, a DNA sequence encoding the target protein, and a DNA sequence encoding a peptide linker and a cleavage site for enterokinase protease, wherein the peptide linker and cleavage site sequence is between the iLOV protein and target protein, and wherein the DNA sequence encoding a fusion protein is configured for introduction in a P. pastoris host ceil to form a P. pastoris recombinant, wherein the P. pastoris recombinant is isolated using fluorescence and the fusion protein is isolated and cleaved with enterokinase to provide the target protein.

The disclosed method further comprising the step of adding at least one specific protein sequence to the iLOV protein to alter properties of the fusion protein.

The disclosed method further comprising the steps of improving production and removing the at least one additional specific protein sequence.

The disclosed method further comprising the steps of simplifying production and removing the at least one additional specific protein sequence scarlessly.

The disclosed method, wherein the step of removing the at least one additional specific protein sequence scarlessly leaves the target protein intact and restore biological/enzymatic activity.

The disclosed method, wherein cleaving the iLOV protein and linker sequences from the target protein comprises using enterokinase.

The disclosed method, wherein identifying an optimal recombinant comprises using a fluorescence to detect the production of heterologous fusion protein.

The disclosed method, further comprising the step of identifying recombinant strains expressing greater than five-fold higher fusion litres compared to randomly selected transformants.

A method tor producing a SARS-CoV-2 vims-like-particle based protein subunit vaccine, the method comprising the steps of creating a fusion protein by combining a DNA sequence encoding an iLOV protein with a DNA sequence encoding a peptide linker and a cleavage site for enterokinase protease and a DNA sequence encoding a Receptor Binding Domain (RBD) of the SARS-CoV-2 viral spike protein, wherein the RBD protein is attached to a “Spy Tag” peptide and the peptide linker cleavage site DNA sequence is between the iLOV protein DNA sequence and one of cither the SARS-CoV-2 viral protein DNA sequence or the “Spy Tag” peptide DNA sequence; introducing a DNA sequence encoding the fusion protein into a P. pastoris host to form transformants; identifying from the transformants at least one optimal recombinant using fluorescence to detect optimal expression levels of the SARS-CoV-2 viral protein; and, isolating the SARS-CoV-2 viral protein from the fusion protein produced by the optimal recombinant by cleaving the iLOV protein and linker sequences from the target protein.

A method for identifying effective metabolite-responsive DNA regulatory regions to produce a target molecule or protein, the method comprising creating mi expression cassette by combining a DNA sequence encoding a reporter iLOV protein or a fusion protein with a microbial metabolite-responsive promotor within a plasmid; introducing a DNA sequence encoding the genetic construct into a host to produce the reporter protein or fusion protein in presence of the metabolite under aerobic or anaerobic conditions: and, identifying an optimal regulatory region for producing the target based on iLOV fluorescence.

The disclosed method, wherein fluorescence of the fusion protein is used to detect impurities throughout the purification of the target protein.

The disclosed method, further comprising an enhanced ability to secrete fusion proteins from P. pastoris to facilitate their purification.

The disclosed method, further comprising the potential to co-express the fusion protein and the enterokinase in the same P. pastoris host, thereby detecting productivity, stability, and enabling maturation of the target protein in the same culture.

The disclosed method, further comprising the potential to co-express and secrete the fusion protein and enterokinase from tire same P. pastoris host, thereby detecting productivity, stability, and enabling maturation of the target protein in the same culture while reducing costs and/or requiring fewer processing steps and simplifying purification.

The disclosed method, further comprising screening codon variations, performance of altered regulatory regions, integrated cassette copy number and/or integration site of tire gene that encodes the target protein to determine the effects) upon productivity and host genetic suability.

The disclosed method, further comprising screening variants of the target protein to determine the effect of mutation(s) upon productivity and host genetic stability.

The disclosed method, further comprising screening homologues of the target protein to determine the effect of natural sequence variation upon productivity and host genetic stability thereby enabling predictability of productivity to help prioritize target proteins for development.

The disclosed method for use with mammalian production hosts such as Chinese hamster ovary (CHO) cells or human embryonic kidney (HEK) cells.

The disclosed method for use with other microbial hosts (where plasmids are available and there should be less heterogeneity) for readily monitoring genetic stability and productivity throughout the process.

The disclosed method for use with other microbial hosts, particularly to screen codon variations or performance of altered regulator regions to produce the target protein and/or determine the effect(s) upon productivity and/or host genetic stability.

The disclosed method, further comprising screening P. pastoris cells for those that have been transformed by and have integrated one or more heterologous DNA fragments without a selectable antibiotic resistance marker.

The disclosed method, further comprising high-throughput identification of E. coli clones showing improved metabolite-induced expression of the iLOV reporter gene or fusion protein when placed under the control of the metabolite-responsive DNA-regulatory region(s).

The disclosed method, further comprising a process where a metabolite is used to induce expression of a fusion protein under control of said metabolite-responsive DNA regulatory region and the fusion protein being comprised of a DNA sequence encoding an iLOV protein, a DNA sequence encoding a peptide linker and a cleavage site for enterokinase protease and a DNA sequence encoding the target protein.

The disclosed method, further comprising the step of adding at least one specific protein sequence to the iLOV protein to alter properties of foe fusion protein.

The disclosed method, wherein cleaving the iLOV protein and linker sequences from the target protein comprises using enterokinase.

The disclosed method, wherein the SARS-CoV-2 RBD protein sequence can be mutated to represent the RBD of SARS-CoV-2 variants (or homologous sequences).

The disclosed method, wherein the SARS-CoV-2 RBD protein sequence can be mutated to improve expression and alter its glycosylation pattern.

The disclosed method, wherein the RBD sequence can belong to any vims within the Coronavirus family.

The disclosed method, wherein the fusion protein combines a DNA sequence encoding an iLOV protein with a DNA sequence encoding a peptide linker and a cleavage site for enterokinase protease and a DNA sequence encoding any viral protein.

The disclosed method, wherein the “Spy Tag” peptide fused to a viral antigen is used in vaccine or diagnostic applications.

The disclosed method, wherein the “Spy Tag” peptide is fused to any recombinantly produced protein or peptide.

The matter set forth in the foregoing description and accompanying drawings is offered by way of illustration only and not as a limitation. While particular embodiments have been shown and described, it will be apparent to those skilled in the art that changes and modifications may be made without departing from the broader aspects of applicants' contribution. The actual scope of the protection sought is intended to be defined in the following claims when viewed in their proper perspective based on the prior art.

The present specification is being filed with a Sequence Listing in accordance with 37 CFR. §§ 1.821 through 1.823. The material of ASCII file titled “Final_Sequencc_Listing.txt” (47 KB), created on Jan. 27, 2021, and submitted via EFS-Web is hereby incorporated by reference.

What is claimed is:

1. A method for isolating optimal host recombinants, the method comprising:
creating a fusion protein by combining a DNA sequence encoding an iLOV protein (reporter protein) with a DNA sequence encoding a peptide linker and a cleavage site for enterokinase protease and a DNA sequence encoding a target protein, wherein the peptide linker DNA sequence is between the iLOV protein DNA sequence and the target protein DNA sequence;
introducing a DNA sequence encoding the fusion protein into a host to form transformants;
identifying from the transformants at least one optimal recombinant using fluorescence to detect optimal expression levels of the target protein; and
isolating the target protein from the fusion protein produced by the optimal recombinant by cleaving the iLOV protein and linker sequences from the target protein.

2. The method of claim 1, wherein the host comprises P. pastoris.

3. The method of claim 1, wherein the host is selected from the group consisting of E. coli, Saccharomyces cerevisiae, Bacillus spp, Pseudomonas putida, Chinese Hamster Ovary (CHO) and Human Embryonic Kidney (HEK).

4. The method of claim 1, wherein the target protein is epidermicin-NI01.

5. The method of claim 1, wherein the target protein comprises a protein having antibacterial activity.

6. The method of claim 2, comprising screening P. pastoris cells for those that have been transformed by and have integrated one or more heterologous DNA fragments without a selectable antibiotic resistance marker.

7. The method of claim 2, further comprising the step of rapidly ranking the productivity of P. pastoris recombinants that express the target protein.

8. The method of claim 2, further comprising the step of rapidly monitoring and ranking the genetic stability of P. pastoris recombinants that express the target protein.

9. The method of claim 1, further comprising the step of selecting a suitable transformant for GMP pharmaceutical manufacture.

10. The method of claim 1, further comprising the step of masking the cytotoxic effects of the target (heterologous) protein to the host cell.

11. The method of claim 2, further comprising the step of masking the cytotoxic effects of the epidermicin-NI01 protein.

12. The method of claim 1, further comprising the step of adding at least one specific additional protein sequence to the iLOV protein to alter properties of the fusion protein and facilitate two-step purification of the target protein.

13. The method of claim 10, further comprising the steps of simplifying production and removing the at least one additional specific protein sequence.

14. The method of claim 10, wherein the step of removing the at least one additional specific protein sequence scarlessly leaves the target protein intact and restores its biological/enzymatic activity.

15. The method of claim 1, wherein cleaving the iLOV protein and linker sequences from the target protein comprises using enterokinase.

16. The method of claim 1, wherein identifying an optimal recombinant comprises using one of either a fluorescence activated cell sorter (FACS) or a fluorescence activated droplet sorter (FADS) to detect the production of heterologous fusion protein.

17. The method of claim 16, further comprising the step of identifying recombinant strains expressing greater than five-fold higher fusion titres compared to randomly selected transformants.

18. The method of claim 1, further comprising the steps of using the iLOV protein as a conditional precipitant, filtering the precipitant to purify the protein, resolubilizing the precipitant.

19. A method for producing a SARS-CoV-2 virus-like-particle based protein subunit vaccine, the method comprising:
creating a fusion protein by combining a DNA sequence encoding an iLOV protein with a DNA sequence encoding a peptide linker and a cleavage site for enterokinase protease and a DNA sequence encoding a Receptor Binding Domain (RBD) of the SARS-CoV-2 viral spike protein, wherein the RBD protein is attached to a “Spy Tag” peptide and the peptide linker cleavage site DNA sequence is between the iLOV protein DNA sequence and one of either the SARS-CoV-2 viral protein DNA sequence or the “Spy Tag” peptide DNA sequence;
introducing a DNA sequence encoding the fusion protein into a P. pastoris host to form transformants;
identifying from the transformants at least one optimal recombinant using fluorescence to detect optimal expression levels of the SARS-CoV-2 viral protein; and
isolating the SARS-CoV-2 viral protein from the fusion protein produced by the optimal recombinant by cleaving the iLOV protein and linker sequences from the target protein.

20. A method for identifying effective metabolite-responsive DNA regulatory regions to produce a target molecule or protein, the method comprising:
creating an expression cassette by combining a DNA sequence encoding one of either a reporter iLOV protein or a fusion protein comprising a DNA sequence encoding an iLOV protein with a DNA sequence encoding a peptide linker and a cleavage site for enterokinase protease and a DNA sequence encoding a target protein, with a microbial metabolite-responsive promotor within a plasmid;
introducing a DNA sequence encoding the genetic construct into a host to produce one of either the reporter protein or the fusion protein in presence of the metabolite under aerobic or anaerobic conditions;
identifying one of either an optimal regulatory region for producing the target or a natural or unnatural metabolite production strain based on iLOV fluorescence.
